SEQUENCE LISTING (29 sequences)

SEQ ID NO: 1 - DNA, 1140 bp, Neisseria gonorrhoeae
ctggcaggcttgacgcttcgatacgctctgtttcggtcaggctggtcccgaaaccggaaaaaccgccgaaaaccaataccctgcatttgagtaaggctgcgctggagagtttcggttcggcggcggcaaagttggaaaaacggcatcccgaattggcggaggcattggcaaacttggttagaaggcatggcgcataaaatgtatacgggaatttgtgtaaacatccgttaatattaagaagtaaaggataatgggtctaatactaaagaaataggttcggggtaaaattgccccttttaaagtaaacgattgtaaacttgcagacaggctttgatttcaaatgaaatttgtagcaaaatgccgccccgaaacatctgtttgtgcaacgcggcggaatctttttcaaggttttgttaatggcggttgcactttgatttctgtaaaaccgaatattattttatcgattggagatttaccatgaaagcttatctggctctgatttctgccgccgttatcggtttggctgcctgctctcaagaacctgccgcgcctgctgccgaggcaactcctgctgctgaagcacccgcttccgaagcgcctgccgccgaagctgctcctgcagatgctgccgaagcccctgctgccggcaattgtgcggcaactgtcgaatccaacgacaatatgcagttcaacaccaaagacatccaagtcagcaaagcatgtaaagagtttaccatcactctgaaacataccggtacgcaacccaaagccagcatgggtcacaaccttgtgattgccaaagctgaagacatggacggcgtatttaaagacggcgtaggtgctgccgataccgactatgtcaaacctgacgatgcgcgcgttgttgcccacaccaaactgatcggcggcggcgaagagtcttccctgactctggatcctgccaaattggctgacggcgactacaaatttgcctgcactttcccgggtcacggtgctttgatgaacggcaaagtgactttggtcgattaatccgcttaaagtctcaaaagacggacagcctgctttgtgcaggctgttttattataaaatgactgcttgaaaagtgccccgttgagaacgaaaacatgaatccgtttgaaa

SEQ ID NO: 2 - DNA, 540 bp, Pseudomonas aeruginosa
ctttttcatgcagcggatcgctcgcgcatcacttcagggtcagggtgcccttcatcagcgcggagtggcccgggaaggtgcagaagaacatgtactgctcgccttccttcagcttggagacgtcgaaggtcaccgagtccttctcgcccgagccgatcagcttggtgtgggcgatgacacggctgtcgtcgggcttcaggtaatccttgtccaggccggaagccatgccgtcggtgaccacgccctgcatgtcggcggcggtgctcagtacccagttgtggcccatgacgttcttcggcaggttgccggggtgggacaggttgacggtgaactgcttgcagctcttgtcgacggtgatggcattggtgttgaactgcatctggtcgttaccctggatgtccaccgagcactcggcagccagcagtggcgcactgagcagggacagcagggataccgcagcgagtttacgtagcatggagcagcctcctaggcaggttgggcgatgaatcctgaaagagcagactgcccgatcgggcaccg

SEQ ID NO: 3 - DNA, 117 bp, Neisseria gonorrhoeae
tgctctcaagaacctgccgcgcctgctgccgaggcaactcctgccggtgaagcacccgcttccgaagcgcctgccgccgaagctgctcctgcagatgctgccgaagcccctgctgcc

SEQ ID NO: 4 - DNA, 33 bp, Artificial Sequence
  Description of Artificial Sequence Synthetic primer
ccggaattccggcagggatgttgtaaatatccg

SEQ ID NO: 5 - DNA, 38 bp, Artificial Sequence
  Description of Artificial Sequence Synthetic primer
ggggtaccgccgtggcaggcatacagcatttcaatcgg

SEQ ID NO: 6 - DNA, 27 bp, Artificial Sequence
  Description of Artificial Sequence Synthetic primer
ggcagcaggggcttcggcagcatctgc

SEQ ID NO: 7 - DNA, 26 bp, Artificial Sequence
  Description of Artificial Sequence Synthetic primer
ctgcaggtcgactctagaggatcccg

SEQ ID NO: 8 - DNA, 25 bp, Artificial Sequence
  Description of Artificial Sequence Synthetic primer
gccgagtgctcggtggacatccagg

SEQ ID NO: 9 - DNA, 27 bp, Artificial Sequence
  Description of Artificial Sequence Synthetic primer
tactcgagtcacttcagggtcagggtg

SEQ ID NO: 10 - DNA, 25 bp, Artificial Sequence
  Description of Artificial Sequence Synthetic primer
cttcagggtcagggtgcccttcatc

SEQ ID NO: 11 - DNA, 26 bp, Artificial Sequence
  Description of Artificial Sequence Synthetic primer
ctgcaggtcgactctagaggatcccg

SEQ ID NO: 12 - DNA, 26 bp, Artificial Sequence
  Description of Artificial Sequence Synthetic primer
tgctctcaagaacctgccgcgcctgc

SEQ ID NO: 13 - DNA, 38 bp, Artificial Sequence
  Description of Artificial Sequence Synthetic primer
taggatccttaggcagcaggggcttcggcagcatctgc

SEQ ID NO: 14 - DNA, 31 bp, Artificial Sequence
  Description of Artificial Sequence Synthetic primer
cgagctcatgtcccctatactaggttattgg

SEQ ID NO: 15 - DNA, 40 bp, Artificial Sequence
  Description of Artificial Sequence Synthetic primer
cccaagctttcaggggatcccacgaccttcgatcagatcc

SEQ ID NO: 16 - DNA, 26 bp, Artificial Sequence
  Description of Artificial Sequence Synthetic primer
ggaattcatatgaaagcttatctggc

SEQ ID NO: 17 - DNA, 27 bp, Artificial Sequence
  Description of Artificial Sequence Synthetic primer
ccggaattcggcagcaggggcttcggc

SEQ ID NO: 18 - DNA, 33 bp, Artificial Sequence
  Description of Artificial Sequence Synthetic primer
cgggatcccctgctctcaagaacctgccgcgcc

SEQ ID NO: 19 - DNA, 41 bp, Artificial Sequence
  Description of Artificial Sequence Synthetic primer
cggaattcttaggcagcaggggcttcggcagcatctgcagg

SEQ ID NO: 20 - DNA, 31 bp, Artificial Sequence; identical to SEQ ID NO: 14
  Description of Artificial Sequence Synthetic primer

SEQ ID NO: 21 - DNA, 32 bp, Artificial Sequence
  Description of Artificial Sequence Synthetic primer
ccgctcgagtcaggcagcaggggcttcggcag

SEQ ID NO: 22 - protein, 166 aa, Neisseria gonorrhoeae
CSQEPAAPAAEATPAGEAPASEAPAAEAAPADAAEAPAAGNCAATVESNDNMQFNTKDIQVSKACKEFTITLKHTGTQPKASMGHNLVIAKAEDMDGVFKDGVGAADTDYVKPDDARVVAHTKLIGGGEESSLTLDPAKLADGDYKFACTFPGHGALMNGKVTLVD

SEQ ID NO: 23 - protein, 128 aa, Pseudomonas aeruginosa
AECSVDIQGNDQMQFNTNAITVDKSCKQFTVNLSHPGNLPKNVMGHNWVLSTAADMQGVVTDGMASGLDKDYLKPDDSRVIAHTKLIGSGEKDSVTFDVSKLKEGEQYMFFCTFPGHSALMKGTLTLK

SEQ ID NO: 24 - protein, 39 aa, Neisseria gonorrhoeae
CSQEPAAPAAEATPAGEAPASEAPAAEAAPADAAEAPAA

SEQ ID NO: 25 - protein, 5 aa, Neisseria gonorrhoeae
AAEAP

SEQ ID NO: 26 - protein, 408 aa, Pseudomonas aeruginosa
MSTEKTKLGVHSEAGKLRKVMVCSPGLAHQRLTPSNCDELLFDDVIWVNQAKRDHFDFVTKMRERGIDVLEMHNLLTETIQNPEALKWILDRKITADSVGLGLTSELRSWLESLEPRKLAEYLIGGVAADDLPASEGANILKMYREYLGHSSFLLPPLPNTQFTRDTTCWIYGGVTLNPMYWPARRQETLLTTAIYKFHPEFANAEFEIWYGDPDKDHGSSTLEGGDVMPIGNGVVLIGMGERSSRQAIGQVAQSLFAKGAAERVIVAGLPKSRAAMHLDTVFSFCDRDLIVPFSLRPDPSSPYGMNIRREEKTFLEVVAESLGLKKLRVVETGGNSFAAEREQWDDGNNVVCLEPGVVVGYDRNTYTNTLLRKAGVEVITISASELGRGRGGGHCMTCPIVRDPIDY

SEQ ID NO: 27 - protein, 151 aa, Pseudomonas aeruginosa
LLTETIQNPEALKWILDRKITADSVGLGLTSELRSWLESLEPRKLAEYLIGGVAADDLPASEGANILKMYREYLGHSSFLLPPLPNTQFTRDTTCWIYGGVTLNPMYWPARRQETLLTTAIYKFHPEFANAEFEIWYGDPDKDHGSSTLEG

SEQ ID NO: 28 - DNA, 30 bp, Artificial Sequence
  Description of Artificial Sequence Synthetic primer
atgcacaatctgctgaccgagaccatccag

SEQ ID NO: 29 - DNA, 27 bp, Artificial Sequence
  Description of Artificial Sequence Synthetic primer
tcaggtcgaggagccgtggtccttgtc

(19) United States
(12) Patent Application Publication
(10) Pub. No.: US 2010/0278937 A1
(43) Pub. Date: Nov. 4, 2010

(54) COMPOSITIONS AND METHODS TO PREVENT AND/OR TREAT CANCER WITH PA-CARD

(76) Inventors: Tapas K. Das Gupta, River Forest, IL (US); Ananda Chakrabarty, Villa Park, IL (US); Arsenio Fialho, Lisbon (PT)

Correspondence Address: DON J. PELTO;Sheppard, Mullin, Richter & Hampton LLP, 1300 I STREET, NW, 11TH FLOOR EAST, WASHINGTON, DC, 20005, US

(21) Appl. No.: 12/709,806

(22) Filed: Feb. 22, 2010

Related U.S. Application Data
(63) Continuation-in-part of application No. 12/314,703, filed on Dec. 15, 2008.
Application No. 12/314,703 is a continuation-in-part of application No. 11/508,173, filed on Aug. 23, 2006.
Application No. 11/508,173 is a continuation-in-part of application No. 11/244,105, filed on Oct. 6, 2005, now Pat. No. 7,691,383.
Application No. 11/244,105 is a continuation-in-part of application No. 11/488,695, filed on Jul. 19, 2006.
(60) Provisional application No. 61/154,236, filed on Feb. 20, 2009.
(60) Provisional application No. 61/179,435, filed on May 19, 2009.

(51) Int. Cl. A61K 33/36; C07K 2/00; C07K 14/21; A61K 38/02; A61K 33/24; A61K 31/497; A61K 31/203; A61K 31/706; C07H 21/04; A61P 35/00; C12N 5/02; C12N 15/63

(52) U.S. Cl. 424/623; 530/300; 530/350; 530/345; 514/19.3; 514/19.4; 514/19.6; 424/649; 514/252.18; 514/559; 514/43; 536/23.5; 435/375; 435/320.1

(57) ABSTRACT

The present invention relates to methods and materials for killing cancer cells with proteins derived from bacteria. The invention specifically relates to Azurin, Laz, Pa-CARD, and fusion proteins Azu-H.8 and H.8-Azu, and their use in killing leukemia cells and/or ovarian cancer cells.

### RELATED APPLICATIONS

This application claims priority under 35 U.S.C. §§119 and 120 to U.S. Provisional Application No. 61/154,236 filed on Feb. 20, 2009 and U.S. Provisional Application No. 61/179,435 filed on May 19, 2009; and is a continuation-in-part of U.S. patent application Ser. No. 12/314,703, filed on Dec. 15, 2008; and is a continuation-in-part of U.S. patent application Ser. No. 11/508,173, filed Aug. 23, 2006; and is a continuation-in-part of U.S. patent application Ser. No. 11/244,105, filed Oct. 6, 2005; and is a continuation-in-part of U.S. patent application Ser. No. 11/488,695, filed Jul. 19, 2006. The entire content of these applications is fully incorporated by reference herein.

The instant application contains a Sequence Listing which has been submitted via EFS-Web and is hereby incorporated by reference in its entirety. Said ASCII copy, created on Apr. 7, 2010, is named 14PR1532.txt and is 15,536 bytes in size.

### BACKGROUND

Leukemia is a malignant cancer of the bone marrow and blood. Characterized by an uncontrolled accumulation of abnormal blood cells, leukemia leads to the inhibition of normal blood cell function and, in many instances, death. It was estimated that leukemia affected approximately 44,270 new people in the United States in 2008. Leukemia is the fifth most common cause of cancer deaths for men and the sixth most common cause of cancer deaths for women. Leukemia causes more deaths than any other cancer among children under age 20. (“Cancer facts and figures 2008,” Atlanta: American Cancer Society (2008); Xie Y et al., Cancer 97:2229-35 (2003)). Certain types of leukemia are extremely difficult to treat—for example, acute promyelocytic leukemia (APL) is a rare but often fatal disease where only 30% of the patients respond to standard chemotherapy.

Many current modes of treatment are effective to a certain extent, but have significant harmful side effects due to lack of specificity toward cancer cells. Efforts to develop target-specific therapies have been greatly spurred by the success of imatinib, a highly effective drug known as Gleevec®. Gleevec® specifically inhibits the Bcr-Abl tyrosine kinase. A chromosome translocation in chronic myeloid leukemia (CML) results in the constitutive activation of the Bcr-Abl tyrosine kinase, which in turn results in the activation of cellular signaling that promotes cancer growth. Retinoic acid, a hormone involved in cellular differentiation, in conjunction with arsenic trioxide (As2O3), are treatments of choice for APL.

However, resistance development is a major problem associated with drugs that target a single point of attack such as a kinase, where conformation-specific binding of the drug can be changed. The increased instances of resistance in leukemia patients has led clinicians to resort to the conventional practice of drug combination therapies, whereby tumor specific drugs are combined to create a synergism of drug activity to allow for the use of smaller doses of nonspecific drugs, and to look for other effective treatments.

### SUMMARY OF THE INVENTION

The present invention relates to compositions and methods of use of cytotoxic peptides to kill cancer, and specifically leukemia and/or ovarian cancer. One aspect of the invention is an isolated peptide that is capable of killing cancer cells, which comprises a caspase recruitment (CARD)-like domain. The isolated peptide may be derived from bacteria, and specifically Pseudomonas aeruginosa. In some embodiments, the isolated peptide is Pa-CARD. In other embodiments, the isolated peptide comprises or consists of SEQ ID NO: 27. In some embodiments, the isolated peptide is capable of killing leukemia, fibrosarcoma, ovarian cancer and/or breast cancer cells. In other embodiments, the isolated peptide is chemically modified to extend or optimize its half-life in the bloodstream.

The present invention also relates to a method of killing cancer cells by contacting the cells with one or more proteins selected from the group consisting of an isolated peptide that is capable of killing cancer cells, which comprises a caspase recruitment (CARD)-like domain, Laz, H8-Azu, and Azu-H8. In some embodiments, the cancer cells are selected from the group consisting of leukemia cells, fibrosarcoma cells, ovarian cancer cells, and breast cancer cells. This method may further comprise contacting the cells with one or more cytotoxic agents that are capable of killing cancer cells. These cytotoxic agents may be, but are not limited to, cisplatin, Gleevec®, Retinoic acid, 5′-aza-2′-deoxycytidine, and arsenic trioxide. In a specific embodiment, the method comprises contacting the cancer cells with cisplatin. In another embodiment, the cancer cells are contacted with the one or more cytotoxic agents at about the same time as the one or more proteins.

The present invention also relates to a method, comprising administering to a mammalian patient suffering from cancer one or more proteins selected from the group consisting of an isolated peptide that is capable of killing cancer cells, which comprises a caspase recruitment (CARD)-like domain, Laz, H8-Azu, and Azu-H8. In some embodiments, the cancer is selected from the group consisting of leukemia, fibrosarcoma, ovarian cancer, and breast cancer. In a further embodiment, one or more cytotoxic agents may that are capable of killing cancer cells may also be administered to the patient. The cytotoxic agents may include, but are not limited to, cisplatin, Gleevec®, Retinoic acid, 5′-aza-2′-deoxycytidine, and arsenic trioxide. In a further embodiment, the additional cytotoxic agent administered to the patient is cisplatin. In another embodiment, the one or more cytotoxic agents are administered at about the same time as the one or more proteins.

The present invention also relates to a method, comprising killing leukemia cells by contacting the cells with an azurin and a peptide comprising the H.8 region of Laz. In a further embodiment, the leukemia cells are contacted with the azurin and the peptide comprising the H.8 region of Laz at or around the same time.

The present invention also relates to a method, comprising administering to a mammalian patient suffering from leukemia an azurin and a peptide comprising the H.8 region of Laz. In a further embodiment, the azurin and the peptide comprising the H.8 region of Laz are administered to the patient at or around the same time.

The present invention also relates to a method, comprising inducing cellular differentiation in a leukemia cell by contacting the leukemia cell with one or more proteins selected from the group consisting of Laz, azurin, H.8-Azu, Azu-H.8, and an isolated peptide that is capable of killing cancer cells, which comprises a caspase recruitment (CARD)-like domain.

The present invention also relates to a method, comprising selectively entering cancer cells by contacting the cancer cells with one or more proteins selected from the group consisting of Laz, azurin, H.8-Azu, Azu-H.8, and an isolated peptide that is capable of killing cancer cells, which comprises a caspase recruitment (CARD)-like domain; wherein the cancer cells are selected from the group consisting of leukemia cells and ovarian cancer cells.

The present invention also relates to a method, comprising inducing cell cycle arrest in a cancer cell by contacting the cancer cell with one or more proteins selected from the group consisting of Laz, azurin, H.8-Azu, Azu-H.8, and an isolated peptide of that is capable of killing cancer cells, which comprises a caspase recruitment (CARD)-like domain. The cancer cell may be selected from a group consisting of a leukemia cell, a fibrosarcoma cell, a breast cancer, and an ovarian cancer cell. In a further embodiment, the protein increases Wee1 protein levels in the cell. In another embodiment, the protein causes the depletion of phosphorylated AKT-Ser-473. In yet another embodiment, the protein both increases Wee1 protein levels in the cell and causes the depletion of phosphorylated AKT-Ser-473.

The present invention also relates to a method, comprising inducing apoptosis in a cancer cell through caspase 3 activation by contacting the cancer cell with an isolated peptide that is capable of killing cancer cells, which comprises a caspase recruitment (CARD)-like domain. In a further embodiment, the cancer cell is an ovarian cancer cell.

The present invention also relates to a method, comprising modulating expression of NF-kB signaling pathway genes in a cancer cell by contacting the cancer cell with an isolated peptide that is capable of killing cancer cells, which comprises a caspase recruitment (CARD)-like domain. In a further embodiment, wherein the cancer cell is an ovarian cancer cell.

The present invention also relates to expression vectors encoding isolated peptides capable of killing that is capable of killing cancer cells, which comprise a caspase recruitment (CARD)-like domain. In a specific embodiment, the expression vector encodes Pa-CARD.

The present invention also relates to pharmaceutical composition comprising an isolated peptide that is capable of killing cancer cells, which comprises a caspase recruitment (CARD)-like domain. In further embodiments, the pharmaceutical composition further comprises a pharmaceutically acceptable carrier. In another embodiment, the pharmaceutical composition further comprises a protein selected from the group consisting of Azurin, Laz, H.8-Azu, and Azu-H.8. In another embodiment, the pharmaceutical composition further comprises one or more cytotoxic agents that are capable of killing cancer cells. In a specific embodiment, the pharmaceutical composition comprises a pharmaceutically acceptable carrier that is appropriate for intravenous injection.

The present invention also relates to a method, comprising administering to a patient suffering from leukemia one or more of the pharmaceutical compositions disclosed in this invention in a therapeutically effective amount. In a further embodiment, the pharmaceutical composition is administered to the patient in a manner selected from the group consisting of intravenously, topically, subcutaneously, intramuscularly, orally, and into a tumor.

Another aspect of the invention is a nucleic acid molecule, which encodes one or more of the isolated peptides disclosed herein.

Another aspect of the invention is a kit comprising one or more of the pharmaceutical compositions described herein.

### BRIEF DESCRIPTION OF THE SEQUENCES

SEQ ID NO: 1 is the genomic DNA coding sequence of the Neisseria gonorrhoeae laz gene, Genbank Accession No. Y00530 (ctggcaggct tgacgcttcg atacgctctg tttcggtcag gctggtcccg aaaccggaaa aaccgccgaa aaccaatacc ctgcatttga gtaaggctgc gctggagagt ttcggttcgg cggcggcaaa gttggaaaaa cggcatcccg aattggcgga ggcattggca aacttggtta gaaggcatgg cgcataaaat gtatacggga atttgtgtaa acatccgtta atattaagaa gtaaaggata atgggtctaa tactaaagaa ataggttcgg ggtaaaattg ccccttttaa agtaaacgat tgtaaacttg cagacaggct ttgatttcaa atgaaatttg tagcaaaatg ccgccccgaa acatctgttt gtgcaacgcg gcggaatctt tttcaaggtt ttgttaatgg cggttgcact ttgatttctg taaaaccgaa tattatttta tcgattggag atttaccatg aaagcttatc tggctctgat ttctgccgcc gttatcggtt tggctgcctg ctctcaagaa cctgccgcgc ctgctgccga ggcaactcct gctgctgaag cacccgcttc cgaagcgcct gccgccgaag ctgctcctgc agatgctgcc gaagcccctg ctgccggcaa ttgtgcggca actgtcgaat ccaacgacaa tatgcagttc aacaccaaag acatccaagt cagcaaagca tgtaaagagt ttaccatcac tctgaaacat accggtacgc aacccaaagc cagcatgggt cacaaccttg tgattgccaa agctgaagac atggacggcg tatttaaaga cggcgtaggt gctgccgata ccgactatgt caaacctgac gatgcgcgcg ttgttgccca caccaaactg atcggcggcg gcgaagagtc ttccctgact ctggatcctg ccaaattggc tgacggcgac tacaaatttg cctgcacttt cccgggtcac ggtgctttga tgaacggcaa agtgactttg gtcgattaat ccgcttaaag tctcaaaaga cggacagcct gctttgtgca ggctgtttta ttataaaatg actgcttgaa aagtgccccg ttgagaacga aaacatgaat ccgtttgaaa).

SEQ ID NO: 2 is the genomic DNA coding sequence of the Pseudomonas aeruginosa azurin gene (ctttttcatg cagcggatcg ctcgcgcatc acttcagggt cagggtgccc ttcatcagcg cggagtggcc cgggaaggtg cagaagaaca tgtactgctc gccttccttc agcttggaga cgtcgaaggt caccgagtcc ttctcgcccg agccgatcag cttggtgtgg gcgatgacac ggctgtcgtc gggcttcagg taatccttgt ccaggccgga agccatgccg tcggtgacca cgccctgcat gtcggcggcg gtgctcagta cccagttgtg gcccatgacg ttcttcggca ggttgccggg gtgggacagg ttgacggtga actgcttgca gctcttgtcg acggtgatgg cattggtgtt gaactgcatc tggtcgttac cctggatgtc caccgagcac tcggcagcca gcagtggcgc actgagcagg gacagcaggg ataccgcagc gagtttacgt agcatggagc agcctcctag gcaggttggg cgatgaatcc tgaaagagca gactgcccga tcgggcaccg).

SEQ ID NO: 3 is the genomic DNA coding sequence of the H.8 region of the Neisseria gonorrhoeae laz gene (tgctctcaag aacctgccgc gcctgctgcc gaggcaactc ctgccggtga agcacccgct tccgaagcgc ctgccgccga agctgctcct gcagatgctg ccgaagcccc tgctgcc).

SEQ ID NO: 4 is the forward primer to PCR amplify the Laz-encoding gene (laz) of Neisseria gonorrhoeae (ccggaattcc ggcagggatg ttgtaaatat ccg).

SEQ ID NO: 5 is the reverse primer to PCR amplify the Laz-encoding gene (laz) of Neisseria gonorrhoeae (ggggtaccgc cgtggcaggc atacagcatt tcaatcgg).

SEQ ID NO: 6 is the forward primer to PCR amplify a 3.1 kb fragment of pUC18-laz (ggcagcaggg gcttcggcag catctgc).

SEQ ID NO: 7 is the reverse primer to PCR amplify a 3.1 kb fragment of pUC18-laz (ctgcaggtcg actctagagg atcccg).

SEQ ID NO: 8 is the forward primer to PCR amplify a 0.4 kb fragment of pUC19-paz (gccgagtgct cggtggacat ccagg).

SEQ ID NO: 9 is the reverse primer to PCR amplify a 0.4 kb fragment of pUC19-paz (tactcgagtc acttcagggt cagggtg).

SEQ ID NO: 10 is the forward primer to PCR amplify a 3.3 kb fragment of pUC19-paz (cttcagggtc agggtgccct tcatc).

SEQ ID NO: 11 is the reverse primer to PCR amplify a 3.3 kb fragment of pUC19-paz (ctgcaggtcg actctagagg atcccg).

SEQ ID NO: 12 is the forward primer to PCR amplify a 0.13 kb fragment of pUC18-laz (tgctctcaag aacctgccgc gcctgc).

SEQ ID NO: 13 is the reverse primer to PCR amplify a 0.13 kb fragment of pUC18-laz (taggatcctt aggcagcagg ggcttcggca gcatctgc).

SEQ ID NO: 14 is the forward primer to PCR amplify the GST-encoding gene from pGEX-5X-3 (cgagctcatg tcccctatac taggttattg g).

SEQ ID NO: 15 is the reverse primer to PCR amplify the GST-encoding gene from pGEX-5X-3 (cccaagcttt caggggatcc cacgaccttc gatcagatcc).

SEQ ID NO: 16 is the forward primer to PCR amplify the signal peptide and H.8-encoding region of laz from pUC18-laz (ggaattcata tgaaagctta tctggc).

SEQ ID NO: 17 is the reverse primer to PCR amplify the signal peptide and H.8-encoding region of laz from pUC18-laz (ccggaattcg gcagcagggg cttcggc).

SEQ ID NO: 18 is the forward primer to PCR amplify the H.8-encoding region from pUC18-laz (cgggatcccc tgctctcaag aacctgccgc).

SEQ ID NO: 19 is the reverse primer to PCR amplify the H.8-encoding region from pUC18-laz (cggaattctt aggcagcagg ggcttcggca gcatctgcag g).

SEQ ID NO: 20 is the forward primer to PCR amplify the GST-H.8 fusion region from pGEX-5X-3-H.8 (cgagctcatg tcccctatac taggttattg g).

SEQ ID NO: 21 is the reverse primer to PCR amplify the GST-H.8 fusion region from pGEX-5X-3-H.8 (ccgctcgagt caggcagcag gggcttcggc ag).

SEQ ID NO: 22 is the amino acid sequence of the Neisseria gonorrhoeae strain F62 Laz protein, Genbank Accession No. Y00530 (Cys Ser Gln Glu Pro Ala Ala Pro Ala Ala Glu Ala Thr Pro Ala Gly Glu Ala Pro Ala Ser Glu Ala Pro Ala Ala Glu Ala Ala Pro Ala Asp Ala Ala Glu Ala Pro Ala Ala Gly Asn Cys Ala Ala Thr Val Glu Ser Asn Asp Asn Met Gln Phe Asn Thr Lys Asp Ile Val Ser Lys Ala Cys Lys Glu Phe Thr Ile Thr Leu Lys His Thr Gly Thr Pro Lys Ala Ser Met Gly His Asn Leu Val Ala Lys Ala Glu Asp Met Asp Gly Val Phe Lys Asp Gly Val Gly Ala Ala Asp Thr Asp Tyr Val Lys Pro Asp Asp Ala Arg Val Val Ala His Thr Lys Leu Ile Gly Gly Gly Glu Glu Ser Ser Leu Thr Leu Asp Pro Ala Lys Leu Ala Asp Gly Asp Tyr Lys Phe Ala Cys Thr Phe Pro Gly His Gly Ala Leu Met Asn Gly Lys Val Thr Leu Val Asp).

SEQ ID NO: 23 is the amino acid sequence of the Pseudomonas aeruginosa azurin (Ala Glu Cys Ser Val Asp Ile Gln Gly Asn Asp Gln Met Gln Phe Asn Thr Asn Ala Ile Thr Val Asp Lys Ser Cys Lys Gln Phe Thr Val Asn Leu Ser His Pro Gly Asn Leu Pro Lys Asn Val Met Gly His Asn Trp Val Leu Ser Thr Ala Ala Asp Met Gln Gly Val Val Thr Asp Gly Met Ala Ser Gly Leu Asp Lys Asp Tyr Leu Lys Pro Asp Asp Ser Arg Val Ile Ala His Thr Lys Leu Gly Ser Gly Glu Lys Asp Ser Val Thr Phe Asp Val Ser Lys Leu Lys Glu Gly Glu Gln Tyr Met Phe Phe Cys Thr Phe Pro Gly His Ser Ala Leu Met Lys Gly Thr Leu Thr Leu Lys).

SEQ ID NO: 24 is the amino acid sequence of the H.8 region from Neisseria gonorrhoeae F62 Laz protein (Cys Ser Gln Glu Pro Ala Ala Pro Ala Ala Glu Ala Thr Pro Ala Gly Glu Ala Pro Ala Ser Glu Ala Pro Ala Ala Glu Ala Ala Pro Ala Asp Ala Ala Glu Ala Pro Ala Ala).

SEQ ID NO: 25 is the amino acid sequence of a peptapeptide motif (Ala Ala Glu Ala Pro).

SEQ ID NO: 26 is the amino acid sequence of P. aeruginosa Arginine Deiminase (ADI) (Met Ser Thr Glu Lys Thr Lys Leu Gly Val His Ser Glu Ala Gly Lys Leu Arg Lys Val Met Val Cys Ser Pro Gly Leu Ala His Gln Arg Leu Thr Pro Ser Asn Cys Asp Glu Leu Leu Phe Asp Asp Val Ile Trp Val Asn Gln Ala Lys Arg Asp His Phe Asp Phe Val Thr Lys Met Arg Glu Arg Gly Ile Asp Val Leu Glu Met His Asn Leu Leu Thr Glu Thr Ile Gln Asn Pro Glu Ala Leu Lys Trp Ile Leu Asp Arg Lys Ile Thr Ala Asp Ser Val Gly Leu Gly Leu Thr Ser Glu Leu Arg Ser Trp Leu Glu Ser Leu Glu Pro Arg Lys Leu Ala Glu Tyr Leu Ile Gly Gly Val Ala Ala Asp Asp Leu Pro Ala Ser Glu Gly Ala Asn Ile Leu Lys Met Tyr Arg Glu Tyr Leu Gly His Ser Ser Phe Leu Leu Pro Pro Leu Pro Asn Thr Gln Phe Thr Arg Asp Thr Thr Cys Trp Ile Tyr Gly Gly Val Thr Leu Asn Pro Met Tyr Trp Pro Ala Arg Arg Gln Glu Thr Leu Leu Thr Thr Ala Ile Tyr Lys Phe His Pro Glu Phe Ala Asn Ala Glu Phe Glu Ile Trp Tyr Gly Asp Pro Asp Lys Asp His Gly Ser Ser Thr Leu Glu Gly Gly Asp Val Met Pro Ile Gly Asn Gly Val Val Leu Ile Gly Met Gly Glu Arg Ser Ser Arg Gln Ala Ile Gly Gln Val Ala Gln Ser Leu Phe Ala Lys Gly Ala Ala Glu Arg Val Ile Val Ala Gly Leu Pro Lys Ser Arg Ala Ala Met His Leu Asp Thr Val Phe Ser Phe Cys Asp Arg Asp Leu Ile Val Pro Phe Ser Leu Arg Pro Asp Pro Ser Ser Pro Tyr Gly Met Asn Ile Arg Arg Glu Glu Lys Thr Phe Leu Glu Val Val Ala Glu Ser Leu Gly Leu Lys Lys Leu Arg Val Val Glu Thr Gly Gly Asn Ser Phe Ala Ala Glu Arg Glu Gln Trp Asp Asp Gly Asn Asn Val Val Cys Leu Glu Pro Gly Val Val Val Gly Tyr Asp Arg Asn Thr Tyr Thr Asn Thr Leu Leu Arg Lys Ala Gly Val Glu Val Ile Thr Ile Ser Ala Ser Glu Leu Gly Arg Gly Arg Gly Gly Gly His Cys Met Thr Cys Pro Ile Val Arg Asp Pro Ile Asp Tyr)

SEQ ID NO: 27 is the amino acid sequence of the CARD region of P. aeruginosa ADI, residues 75-225 (Leu Leu Thr Glu Thr Ile Gln Asn Pro Glu Ala Leu Lys Trp Ile Leu Asp Arg Lys Ile Thr Ala Asp Ser Val Gly Leu Gly Leu Thr Ser Glu Leu Arg Ser Trp Leu Glu Ser Leu Glu Pro Arg Lys Leu Ala Glu Tyr Leu Ile Gly Gly Val Ala Ala Asp Asp Leu Pro Ala Ser Glu Gly Ala Asn Ile Leu Lys Met Tyr Arg Glu Tyr Leu Gly His Ser Ser Phe Leu Leu Pro Pro Leu Pro Asn Thr Gln Phe Thr Arg Asp Thr Thr Cys Trp Ile Tyr Gly Gly Val Thr Leu Asn Pro Met Tyr Trp Pro Ala Arg Arg Gln Glu Thr Leu Leu Thr Thr Ala Ile Tyr Lys Phe His Pro Glu Phe Ala Asn Ala Glu Phe Glu Ile Trp Tyr Gly Asp Pro Asp Lys Asp His Gly Ser Ser Thr Leu Glu Gly)

SEQ ID NO: 28 is the nucleotide sequence of a forward primer to PCR amplify the CARD motif of the P. aeruginosa ADI gene (ATGCACAATC TGCTGACCGA GACCATCCAG).

SEQ ID NO: 29 is the nucleotide sequence of a reverse primer to PCR amplify the CARD motif of the P. aeruginosa ADI gene (CAGGTCGAGG AGCCGTGGTC CTTGTC).

### BRIEF DESCRIPTION OF THE FIGURES

FIG. 1. FIG. 1 depicts a schematic representation of laz from Neisseria gonorrhoeae (A) and paz from Pseudomonas aeruginosa (B). The P. aeruginosa azurin gene for cloning and hyperexpression in E. coli consisted of the azurin gene itself (paz) and the signal peptide (psp) sequence that determines its periplasmic location (B). The H.8 region of laz was cloned in frame either in the 5′-end of the paz gene (C) including the Neisserial signal sequence nsp (pUC18-H.8-paz) or at the 3′-end of the paz gene (D) (pUC19-paz-H.8). The detailed procedures for preparing the constructs are given in Example 1. naz, azurin-like sequence of Neisseria gonorrhoeae present in the laz gene; nsp, Neisseria signal peptide sequence. The signal peptide sequence in both cases is cleaved off to produce the mature Paz (periplasmic) and Laz (surface-exposed) proteins. (E), SDS-PAGE of Laz, Paz and the fusion proteins. The anomalous migration of the H.8 fusion proteins such as Laz, H.8-Paz or Paz-H.8 (all about 17 kDa) has previously been noted for lapidated H.8-containing proteins (Cannon, Clin. Microbiol. Rev. 2:S1-S4 (1989); Fisette, et al., J. Biol. Chem. 278:46252-46260 (2003)).

FIG. 2. FIG. 2 depicts graphs illustrating the degree to which the H.8-Paz fusion proteins are cytotoxic to various cancer cells. (A) Cytotoxicity of synthetic H.8 peptide, Paz, Laz and H.8 fusions at the carboxy terminal end of Paz (Paz-H.8) and amino terminal end of the Paz (H.8-Paz) towards glioblastoma LN-229 cells. Cells were treated with the proteins at 3 different concentrations (10, 20 and 40 μM) for 6, 12 and 24 h. MTT assay was done to measure the extent of live cells to account for cytotoxicity (percent cell death). To calculate percentage cytotoxicity, the value of non-treated viable cells was taken to be 100% and the number of viable cells was determined in Paz, Laz and H.8-fusion protein-treated samples. The extent of cytotoxicity (%) was then determined from the number of dead cells. (B) Cytotoxicity of H.8 peptide, Paz, Paz-H.8, H.8-Paz and Laz towards human breast cancer MCF-7 cells. All treatment conditions are similar to (A) above.

FIG. 3. FIG. 3 depicts the entry of various fluorescently labeled azurin-related proteins into glioblastoma LN-229 and breast cancer MCF-7 cells. (A) H.8 peptide, Paz, Paz-H.8, H.8-Paz and Laz (20 μM each) conjugated with Alexa fluor® 568 was incubated with LN-229 cells on a coverslip at 37° C. for 30 min after which images were taken. (B) Internalization into MCF-7 cells of various proteins conjugated with Alexa fluor® 568 as visualized by confocal microscopy and as described for (A). (C) Internalization of Laz was visualized by confocal microscopy. Various concentrations (2, 4, 8 and 16 μM) of fluorescently-labeled Laz were incubated with LN-229 cells for 30 min at 37° C. The nucleus is labeled blue with DAPI (4,6-diamidino-2-phenylindole). (D) Laz (10 μM) conjugated with Alexa fluor® 568 was incubated with LN-229 cells for various time periods (5, 10, 20 and 30 min) at 37° C. The internalization was visualized by confocal microscopy. (E) Paz (10 μM) conjugated with Alexa fluor® 568 was incubated with LN-229 cells on a coverslip at 37° C. for various times after which images were taken. Very little measurable fluorescence was detected in (E).

FIG. 4. FIG. 4 depicts bar graphs indicating the quantification of the fluorescence found in the confocal microscope images in FIG. 3A-D. (A) Quantification of fluorescence in images in FIG. 3A. Quantification of fluorescence in azurin proteins was done by using Adobe® Photoshop®. Error bars represent the standard deviation of the fluorescence in three different cells in a single sample. (B) Quantification of fluorescence in images in FIG. 3B. Quantification performed as in FIG. 4A. (C) Quantification of fluorescence in images in FIG. 3C. Quantification performed as in FIG. 4A. (D) Quantification of fluorescence in images in FIG. 3D. Quantification performed as in FIG. 4A.

FIG. 5. FIG. 5 depicts images and a graph of uptake and cytotoxicity, respectively, of labeled fusion proteins in cells. Combined treatment with H.8-GST fusion proteins facilitates the uptake of Alexa fluor® 568-labeled Paz in glioblastoma LN-229 cells. Unlabeled 20 μM (A) H.8, (B) GST, (C) GST-H.8, (D) H.8-GST, (E) PBS buffer and 20 μM Paz conjugated with Alexa fluor® 568 were incubated with LN-229 cells for 30 min at 37° C. The internalization was visualized by confocal microscopy. (F) Cytotoxicity of synthetic H.8 peptide, GST and GST-H.8/H.8-GST fusion derivatives with or without Paz. Approximately 5×10³ LN-229 cells were seeded into 96-well culture plate and treated with 20 μM each of H.8 peptide, GST, GST-H.8, H.8-GST or the same volume of PBS buffer for 24 h with (+Paz) or without (−Paz) 20 μM Paz.

FIG. 6. FIG. 6 depicts images of the brains of mice injected with Paz, H.8-Paz and Laz conjugated with IRdye® 800CW (LI-COR Biotechnology, Lincoln, Nebr.). (A) Brain images from live mice. Five hundred μg of Paz, H.8-Paz and Laz conjugated with IRdye® 800CW were injected intraperitoneally in live nude mice. After 24 h, the mice were sacrificed, brains were taken out and the fluorescence was detected and measured with the LI-COR Odyssey® Infrared Imaging System. (B) Rostral mesencephalon region images of nude mice brains treated as in (A). Mice brains were cut horizontally and images were taken.

FIG. 7. FIG. 8 depicts SDS-PAGE, Western blotting and confocal microscope images of localization of H.8-Gst fusion proteins in E. coli. (A). E. coli BL21 (DE3) cells having cloned gst, H.8-gst or gst-H.8 genes were cultured at 37° C. with 0.1 mM IPTG. Cell pellets were washed with PBS twice, and whole cell lysates were run on SDS-PAGE. Coomassie blue staining was used for detection of the proteins. (B). The above procedure was repeated but this time both whole cell lysates and the contents of the periplasmic space were separately isolated, run on SDS-PAGE (20 μg protein) and the GST or GST-H.8 fusion proteins were detected by Western blotting with monoclonal anti-GST antibody to determine the total and the periplasmic concentrations of the proteins. (C). E. coli strain BL21(DE) cells harboring cloned gst, H.8-gst or gst-H.8 genes (Table 2) were cultured at 37° C. with 0.4 mM IPTG. One ml each of these bacterial cultures were centrifuged and the resultant bacterial pellets were collected. After washing with PBS twice, one ml of 1% FBS-PBS containing anti-GST antibody (1:2000) was applied. Cell suspensions were incubated for 1 h and then washed with PBS twice. Bacterial cells were incubated with FITC-conjugated anti-rabbit IgG in 1% FBS-PBS for 30 min. To remove unbound antibody, cells were washed again, and fixed with ethanol on ice. E. coli samples treated with DAPI (imparting blue coloration) were observed under confocal microscopy (×100 objective), and a single cell was also photographed. (D). E. coli cells harboring pUC19-paz (P. aeruginosa azurin), pUC19-laz (Neisseria), pUC18-H.8-paz or pUC18-paz-H.8 were cultured at 37° C. overnight in presence of 0.1 mM IPTG. 0.5 ml of such cultures were centrifuged and the resultant bacterial pellets were washed with chilled PBS twice. Anti-azurin antibody (1:500) in 1 ml of 1% FBS-PBS was applied and incubated on ice for 1 h. After washing with PBS twice, FITC-conjugated anti-rabbit antibody was applied, incubated on ice for 30 min, washed with PBS twice and fixed with cold ethanol. Bacterial samples were observed by confocal microscopy (×100 objective).

FIG. 8. FIG. 8 depicts graphs showing cell cytotoxicity as determined by MTT assay. (A and B) Cell cytotoxicity was determined by MTT assay of HL60 (A) and K562 (B) cells treated with 10 μM of Laz, Azurin (Azu), H8-Azu, and Azu-H8 or 5 μM DAC as a positive control after 24 h, 48 h and 72 h. (C) K562 cells treated with low concentrations: 3.75 nM, 37.5 nM, 75 nM, 2.5 μM, 5 μM and 10 μM of Laz, Azu, H8-Azu, Azu-H8 or 5 μM DAC as a positive control. (D) Cell cytotoxicity determined by MTT assay of HL60 cells treated with varying concentrations 1 μM, 2.5 μM, 5 μM and 10 μM of Laz, Azu, H8-Azu, Azu-H8 or 5 μM DAC. Data are representative of the mean (±standard error) of three separate experiments done in triplicate. All values are significantly different from controls (p<0.05).

FIG. 9. FIG. 9 depicts fluorescent microscopy images of changes in cell morphology induced by Laz and Azurin in HL60 and K562 cells. Laz induces morphological changes in HL60 and K562 cells as shown by fluorescent microscopy. (A) and (B) represent untreated HL60 and K562 cells. HL60 (C) or K562 (D) cells treated with 10 μM Laz for 48 h undergo differentiation, a process that is linked to subsequent cellular apoptosis. Arrows point to differentiated granulomatous cells. Fewer morphological changes are seen in HL60 cells (E) or K562 cells (F) treated with 10 μM Azurin for 48 h, even though the cytotoxicity was high.

FIG. 10. FIG. 10 depicts images from confocal microscopy showing the selective entry of Laz and Azurin into HL60 and K562 cells. Panel 1: Neither Azurin nor Laz enters normal peripheral blood mononuclear cells (PBMCs) after 1 h incubation at 10 μM concentration (A and B). Azurin enters K562 cells after 1 h incubation (D), as does Laz (C). Azurin enters HL60 cells after 1 h incubation (F), as does Laz (E). Panel 2: In comparison with control K562 cells, Azu-H8, H8-Azu enter such K562 cells as well as Laz.

FIG. 11. FIG. 11 depicts graphs charting of cell cycle progression in the absence and in the presence of Azurin, Laz and DAC. K562 cells were treated for 48 h with 10 μM Laz, Azurin and 5 μM DAC, fixed, stained and analyzed for DNA content as described previously.

FIG. 12. FIG. 12 depicts images of immunoblots. This figure provides analysis of the status of phosphorylation of AKT (AKT-P-Ser473) and Wee1 protein levels via immunoblotting of proteins in Laz- and Pa-CARD-treated K562 and HL-60 cells in cytosolic and nuclear extracts. Western blot analysis of cytosolic and nuclear proteins comparing levels of Wee1, and phospho-AKT serine 473 from HL60 and K562 cells after 48 h of treatment with 10 μM Laz or 10 uM Pa-CARD compared to a control of untreated cells is shown.

FIG. 13. FIG. 13 depicts a schematic representation of the CARD domain of ADI from P. aeruginosa that has cytotoxic activity against leukemia cells. (A) The presence of the CARD domain in Pa-ADI and Ma-ADI, but its absence in other members of the guanidino-group modifying superfamily enzymes such as DDAH and AGAT, as shown from the structure-based sequence alignments. The CARD domain represents that of mammalian caspase-9. DDAH, dimethylarginine dimethylaminohydrolase; AGAT, arginine: glycine amidinotransferase. The β-strand and α-helices are shown as arrows and strips, respectively. Roman numerals I to V are the five ββαβ subunits. The clip moiety (α-helical module) is seen in Pa-ADI and in M. arginini ADI (Ma-ADI) which bears structural homology to the six-α-helical modules that form the CARD domain of the caspase-9 protein molecule. N,N-terminal; C,C-terminal. (B) SDS-PAGE gel migration of purified 46 kDa ADI and 17 kDa CARD proteins from P. aeruginosa. (C) Cytotoxic activity of Pa-ADI and Pa-CARD on fibrosarcoma (HT-1080), breast cancer (MCF-7) and leukemia (HL60) cells. Cells were incubated with 10 μM Pa-ADI or Pa-CARD for 48 h, after which cell viability was determined by MTT assays.

FIG. 14. FIG. 14 depicts images showing structural characteristics of the arginine deiminase (ADI) enzymes from Mycoplasma arginini and Pseudomonas aeruginosa. (A) Ribbon drawing of P. aeruginosa ADI (1RXX_A) highlighting the two domains (MolMol program). (B) A copy of the image shown in FIG. 13A herein. (C) A schematic diagram showing the overall fold and topology of P. aeruginosa ADI (Pa-ADI), as described.

FIG. 15. FIG. 15 depicts gel images and a graph showing the purification and arginine deiminase enzymatic activity of Pa-ADI and Pa-CARD. (A) SDS-PAGE gel migration of purified ADI and CARD proteins from P. aeruginosa. (B) The kinetics of ADI activity at 37° C. and pH 7.2 of the purified Pa-ADI and Pa-CARD. The product of the enzymatic reaction, citrulline, was measured at 490 nm. No arginine deiminase enzymatic activity of Pa-CARD was detected.

FIG. 16. FIG. 16 depicts graphs showing that Pa-CARD has higher cytotoxicity than Pa-ADI against a range of cancer cells. (A) Fibrosarcoma (HT-1080), breast cancer (MCF-7) and ovarian cancer (SKOV-3) cells were incubated with 20 μM Pa-ADI or Pa-CARD for 48 hours, after which cell viability was determined by MTT assays. The mean (±standard error) of the number of viable cells per well was determined from 3 experiments in triplicate cultures. All values are significantly different from controls (p<0.05). (B) Dose dependent cytotoxicity exhibited by Pa-CARD towards the ovarian cancer SKOV-3 cells as a function of time (24, 48, and 72 hours). (C) The same experiments were conducted with Pa-ADI. The mean (±standard error) of the number of viable cells per well was determined from 3 experiments in triplicate cultures as described in A (p<0.05).

FIG. 17. FIG. 17 depicts graphs showing the comparative cytotoxicity effect of Pa-CARD, azurin and cisplatin in ovarian cancer SKOV-3 cells. (A) Pa-ADI, Pa-CARD, azurin, and cisplatin, all at 20 μM, were incubated with either SKOV-3 or the normal ovarian HOSE6-3 cells for 48 hours, after which cell viability was determined by MTT assay. (B) Pa-CARD shows some additive effect with cisplatin compared to individual treatment with SKOV-3 cells.

FIG. 18. FIG. 18 depicts photographs showing that Pa-CARD induces apoptosis in SKOV-3 cells but not in HOSE6-3 cells through caspase activation. The TUNEL assay was used to detect apotosis-induced DNA strand breaks by measuring the incorporation of flourescein-tagged dUTP using the in situ Cell Death Detection Fluorescein kit (Roche). SKOV-3 and HOSE6-3 cells were grown in 8 well Lab Tek chamber slides and incubated with 10 μM of Pa-CARD, Pa-ADI and azurin for 48 hours. A negative control without treatment or with BSA was also maintained in parallel (bottom row). The nucleus of the cells was stained blue with DAPI. Cells viewed under green (A) and blue (B) channels as well as the superimposed images (C) (Cyan color) are shown.

FIG. 19. FIG. 19 depicts a graph showing caspase activation by Pa-CARD. Caspase activity was measured by Caspase-Glo™ 3/7 assay kit purchased from Promega. Pa-ADI and Pa-CARD were used at two different concentrations to evaluate the dose response effect. Increase in caspase activity is expressed as fold increase as compared to a control without a drug treatment.

### DETAILED DESCRIPTION OF THE EMBODIMENTS

### Definitions

As used herein, the term “cell” includes both the singular or the plural of the term, unless specifically described as a “single cell.”

As used herein, the term “cytotoxic peptide” means peptides of the present invention that are selectively cytotoxic to cancer cells, and specifically leukemia cells, but not normal cells.

As used herein, the terms “polypeptide,” “peptide,” and “protein” are used interchangeably to refer to a polymer of amino acid residues. The terms apply to amino acid polymers in which one or more amino acid residue is an artificial chemical analogue of a corresponding naturally occurring amino acid. The terms also apply to naturally occurring amino acid polymers. The terms “polypeptide,” “peptide,” and “protein” are also inclusive of modifications including, but not limited to, glycosylation, lipid attachment, sulfation, gamma-carboxylation of glutamic acid residues, hydroxylation and ADP-ribosylation. It will be appreciated that polypeptides are not always entirely linear. For instance, polypeptides may be branched as a result of ubiquitination and they may be circular (with or without branching), generally as a result of post-translation events, including natural processing event and events brought about by human manipulation which do not occur naturally. Circular, branched and branched circular polypeptides may be synthesized by non-translation natural process and by entirely synthetic methods as well. A synthetic peptide is one made without the aid of cellular components. Synthetic methods to make peptides are well known in the art and are commercially available. Further, this invention contemplates the use of both the methionine-containing and the methionine-less amino terminal variants of the protein of the invention.

As used herein, the term “condition” includes anatomic and physiological deviations from the normal that constitute an impairment of the normal state of the living animal or one of its parts, that interrupts or modifies the performance of the bodily functions.

As used herein, the term “inhibit cell growth” means the slowing or ceasing of cell division and/or cell expansion. This term also includes the inhibition of cell development or increases in cell death.

As used herein, the term “suffering from” includes presently exhibiting the symptoms of a condition, having a condition even without observable symptoms, in recovery from a condition, and recovered from a condition.

A used herein, the term “treatment” includes preventing, lowering, stopping, or reversing the progression or severity of the condition or symptoms associated with a condition being treated. As such, the term “treatment” includes medical, therapeutic, and/or prophylactic administration, as appropriate. Treatment may also include preventing or lessening the development of a condition, such as premalignant lesions or cancer.

A “therapeutically effective amount” is an amount effective to prevent, lower, stop or reverse the development of, or to partially or totally alleviate the existing symptoms of a particular condition for which the subject being treated. Determination of a therapeutically effective amount is well within the capability of those skilled in the art.

The term “substantially pure”, as used herein, when used to modify a protein or other cellular product of the invention, refers to, for example, a protein isolated from the growth medium or cellular contents, in a form substantially free of, or unadulterated by, other proteins and/or active inhibitory compounds. The term “substantially pure” refers to a factor in an amount of at least about 75%, by dry weight, of isolated fraction, or at least “75% substantially pure.” More specifically, the term “substantially pure” refers to a compound of at least about 85%, by dry weight, active compound, or at least “85% substantially pure.” Most specifically, the term “substantially pure” refers to a compound of at least about 95%, by dry weight, active compound, or at least “95% substantially pure.” The term “substantially pure” may also be used to modify a synthetically made protein or compound of the invention, where, for example, the synthetic protein is isolated from the reagents and by-products of the synthesis reaction(s).

The term “pharmaceutical grade”, as used herein, when referring to a peptide or compound of the invention, is a peptide or compound that is isolated substantially or essentially from components which normally accompany the material as it is found in its natural state, including synthesis reagents and by-products, and substantially or essentially isolated from components that would impair its use as a pharmaceutical. For example, a “pharmaceutical grade” peptide may be a isolated from any carcinogen. In some instances, “pharmaceutical grade” may be modified by the intended method of administration, such as “intravenous pharmaceutical grade,” in order to specify a peptide or compound that is substantially or essentially isolated from any substance that would render the composition unsuitable for intravenous administration to a patient. For example, an “intravenous pharmaceutical grade” peptide may be isolated from detergents, such as SDS, and anti-bacterial agents, such as azide.

The phrases “isolated,” “purified” or “biologically pure” refer to material which is substantially or essentially free from components which normally accompany the material as it is found in its native state. Thus, isolated peptides in accordance with the invention preferably do not contain materials normally associated with the peptides in their in situ environment. An “isolated” region refers to a region that does not include the whole sequence of the polypeptide from which the region was derived. An “isolated” nucleic acid, protein, or respective fragment thereof has been substantially removed from its in vivo environment so that it may be manipulated by the skilled artisan, such as but not limited to nucleotide sequencing, restriction digestion, site-directed mutagenesis, and subcloning into expression vectors for a nucleic acid fragment as well as obtaining the protein or protein fragment in substantially pure quantities.

The term “variant” as used herein with respect to a peptide, refers to amino acid sequence variants which may have amino acids replaced, deleted, or inserted as compared to the wild-type polypeptide. Variants may be truncations of the wild-type peptide. A “deletion” is the removal of one or more amino acids from within the wildtype protein, while a “truncation” is the removal of one or more amino acids from one or more ends of the wildtype protein. Thus, a variant peptide may be made by manipulation of genes encoding the polypeptide. A variant may be made by altering the basic composition or characteristics of the polypeptide, but not at least some of its fundamental activities. For example, a “variant” of a Neisseria peptide or a Pa-CARD peptide may be a mutated Neisseria peptide or Pa-CARD peptide that retains its ability to kill leukemia cells. In some cases, a variant peptide is synthesized with non-natural amino acids, such as ε-(3,5-dinitrobenzoyl)-Lys residues. (Ghadiri & Fernholz, J. Am. Chem. Soc., 112:9633-9635 (1990)). In some embodiments, the variant has not more than 20, 19, 18, 17 or 16 amino acids replaced, deleted or inserted compared to wild-type peptide. In some embodiments, the variant has not more than 15, 14, 13, 12 or 11 amino acids replaced, deleted or inserted compared to wild-type peptide. In some embodiments, the variant has not more than 10, 9, 8 or 7 amino acids replaced, deleted or inserted compared to wild-type peptide. In some embodiments, the variant has not more than 6 amino acids replaced, deleted or inserted compared to wild-type peptide. In some embodiments, the variant has not more than 5 or 4 amino acids replaced, deleted or inserted compared to wild-type peptide. In some embodiments, the variant has not more than 3, 2 or 1 amino acids replaced, deleted or inserted compared to wild-type peptide. In some embodiments, the variant has been created using the techniques and methods described in U.S. patent application Ser. No. 12/389,120, the disclosure of which is incorporated by reference in its entirety herein.

The term “amino acid,” as used herein, means an amino acid moiety that comprises any naturally-occurring or non-naturally occurring or synthetic amino acid residue, i.e., any moiety comprising at least one carboxyl and at least one amino residue directly linked by one, two, three or more carbon atoms, typically one (a) carbon atom. The term “residue” is synonymous with “amino acid.”

The term “derivative” as used herein with respect to a peptide refers to a peptide that is derived from the subject peptide. A derivation includes chemical modifications of the peptide such that the peptide still retains some of its fundamental activities. For example, a “derivative” of a Neisseria peptide or a Pa-CARD peptide can be a chemically modified Neisseria peptide or Pa-CARD peptide that retains its ability to kill leukemia and/or ovarian cancer cells. Chemical modifications of interest include, but are not limited to, amidation, acetylation, sulfation, polyethylene glycol (PEG) modification, phosphorylation or glycosylation of the peptide. In addition, a derivative peptide maybe a fusion of a polypeptide or fragment thereof to a chemical compound, such as but not limited to, another peptide, drug molecule or other therapeutic or pharmaceutical agent or a detectable probe. In some embodiments, the derivative has been created using the techniques and methods described in U.S. patent application Ser. No. 12/389,120, the disclosure of which is incorporated by reference in its entirety herein.

The term “percent (%) amino acid sequence identity” is defined as the percentage of amino acid residues in a polypeptide that are identical with amino acid residues in a candidate sequence when the two sequences are aligned. To determine % amino acid identity, sequences are aligned and if necessary, gaps are introduced to achieve the maximum % sequence identity; conservative substitutions are not considered as part of the sequence identity. Amino acid sequence alignment procedures to determine percent identity are well known to those of skill in the art. Often publicly available computer software such as BLAST, BLAST2, ALIGN2 or Megalign (DNASTAR) software is used to align peptide sequences. In a specific embodiment, Blastp (available from the National Center for Biotechnology Information, Bethesda Md.) is used using the default parameters of long complexity filter, expect 10, word size 3, existence 11 and extension 1.

When amino acid sequences are aligned, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) can be calculated as:

% amino acid sequence identity=X/Y*100

where
X is the number of amino acid residues scored as identical matches by the sequence alignment program's or algorithm's alignment of A and B and
Y is the total number of amino acid residues in B.
   If the length of amino acid sequence A is not equal to the length of amino acid sequence B, the % amino acid sequence identity of A to B will not equal the % amino acid sequence identity of B to A. When comparing longer sequences to shorter sequences, the shorter sequence will be the “B” sequence. For example, when comparing truncated peptides to the corresponding wild-type polypeptide, the truncated peptide will be the “B” sequence.

### General

The present invention provides compositions comprising bacterial peptides, and variants, derivatives, and structural equivalents of such peptides, that have cytotoxic effects on cancer cells but not normal cells, and methods to prevent the development of cancer in mammals, treat cancer in mammals, and kill mammalian cancer cells. Specifically, the compositions and methods disclosed herein can be used to prevent, treat, and/or kill leukemia and/or ovarian cancer.

It has been shown that many cupredoxin proteins, particularly Pseudomonas aeruginosa azurin and truncations thereof, have the ability to preferentially enter and kill many types of solid mammalian cancer cells both in vivo and in vitro. (Yamada et al., Cell. Biol. 7:1418-1431 (2005); Hiraoka et al., PNAS 101:6427-6432 (2004); Hiraoka et al., Biochem. Biophys. Res. Comm. 338:1284-1290 (2005)). See also U.S. Pat. Nos. 7,491,394, 7,381,701, and 7,084,105 and U.S. patent application Ser. Nos. 11/488,693, 11/950,165, 11/854,654, and 12/338,480, the disclosures of which are incorporated by reference in their entirety herein. An azurin-like gene exists in many gonococci and meningococci, such as Neisseria gonorrhoeae and N. meningitidis. (Gotschlich & Seiff, FEMS Microbiol. Lett. 43:253-255 (1987); Kawula, et al., Mol. Microbiol. 1:179-185 (1987)). Azurin is produced by a number of pathogenic bacteria and there is significant sequence homology among such genes. (Yamada, et al., Cell. Microbiol. 7:1418-1431 (2005))

A protein epitope termed “H.8” is conserved among pathogenic Neisseria species and is detected by the binding of a monoclonal antibody designated H.8. The distinct gonococcal gene laz encodes a protein that cross-reacts with the H.8 monoclonal antibody. (Hayashi & Wu, J. Bioenerg. Biomembr. 22:451-471 (1990)).

Laz is a gonococcal outer surface protein that contains a signal peptide lipoprotein consensus sequence that is recognized by the bacterial enzyme signal peptidase II, which processes the sequence to result in the N-terminal acylation of a cysteine residue with fatty acid and glycerol. (Hayashi & Wu, id.; Yamada, et al., Cell. Microbiol. 7:1418-1431 (2005)). The Laz lipoprotein, about 17 kDa, includes an H.8 region that is a 39 amino acid region at the N-terminus containing imperfect pentapeptide repeats of the motif Ala-Ala-Glu-Ala-Pro (AAEAP (SEQ ID NO: 25)). (Gotschlich & Seiff, id.; Kawula, et al., id.; Woods et al., Mol. Microbiol. 3: 43-48 (1989)). Beyond this 39 amino acid N-terminal region in Laz is a 127 amino acid region that is highly homologous to P. aeruginosa azurin. (Cannon, Clin. Microbiol. Rev. 2:S1-S4 (1989)) Laz is involved in defense against oxidative stress and copper toxicity and increases survival in an ex vivo primary human ectocervical epithelial assay. (Wu, et al., Infect. Immun. 73:8444-8448 (2005)).

It is now known that the Laz protein, the azurin-like protein from Neisseria gonorrhoeae and other Neisseria species, is able to specifically enter and kill brain cancer cells such as glioblastoma cells, as well as other tumors. See Examples 2 and 7. Furthermore, it is now known that the H.8 region of the Laz protein can confer upon P. aeruginosa azurin when fused to either its N-terminal or C-terminal, the ability to enter and kill glioblastoma cells. See Examples 2 and 3.

It is also now known that the H.8 region does not have to be physically attached to a co-administered protein, such as azurin, to confer upon that protein the ability to enter glioblastoma cells. See Example 5. H.8 and H.8 fused to the N-terminus of GST both increased the entry of physically unattached azurin into glioblastoma cells as compared to azurin alone, however H.8 fused to the C-terminus of GST was ineffective. Further, the H.8 and H.8 fused to the N-terminal of GST when coadministered with azurin both enhanced the cytotoxicity of azurin towards glioblastoma cells. See Example 5. It is also contemplated that this AAEAP (SEQ ID NO: 25) repeat unit can be used to design peptides that will kill leukemia cells.

Azurin and laz are known to target multiple steps in the cancer progression pathway, such as induction of apoptosis by complex formation and stabilization of p53, inhibition of angiogenesis, as well as inhibition of cell signaling mediated by receptor tyrosine kinases such as EphB2/ephrin B2, thereby minimizing the chances of resistance development. See, e.g., U.S. Pat. No. 7,381,701, the disclosure of which is incorporated by reference in its entirety herein. Azurin and Laz are known to have entry specificity in melanoma or breast cancer cells, as compared to normal cells.

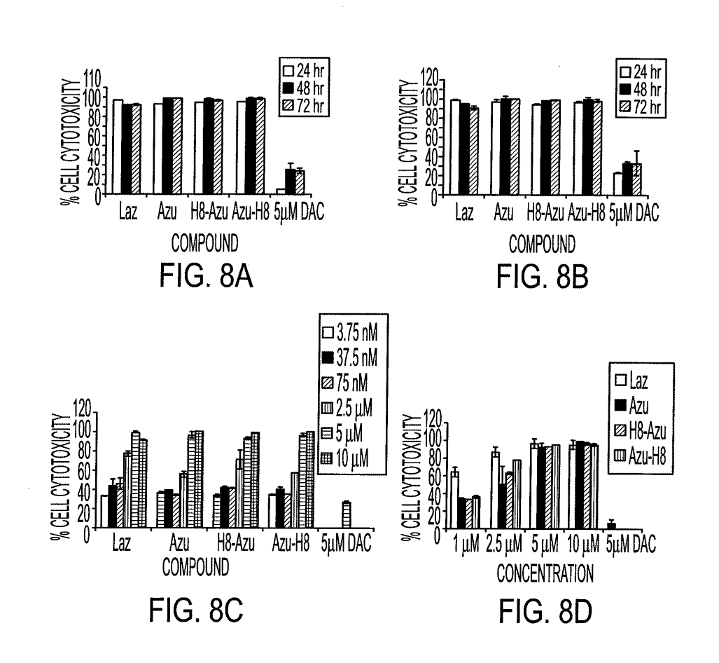

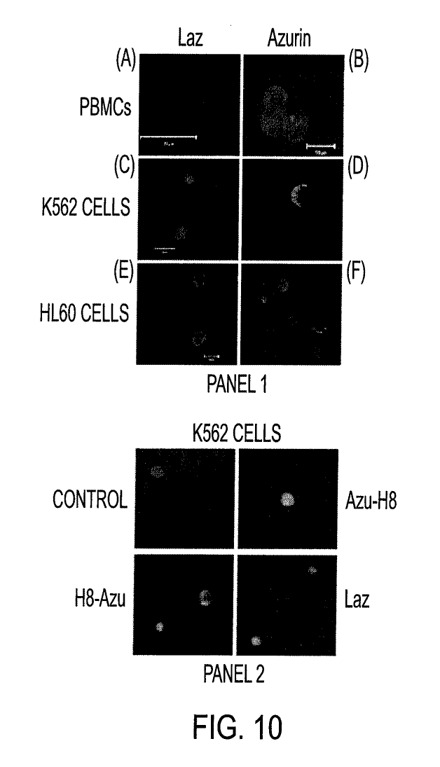

Surprisingly, in addition to being cytotoxic to solid-type cancer cells, the Azurin and Laz proteins are also effective against liquid-borne cancers such as leukemia, as demonstrated by the Examples set forth herein. The Examples demonstrate that Azurin and Laz can each enter leukemia cell lines and have cytotoxic effects therein. Furthermore, as discussed in Examples 9 and 11 and demonstrated in FIGS. 8-11, Laz exerts cytotoxic effect on leukemia cell lines while having little effect on normal peripheral blood mononuclear cells (PBMCs), where they have very limited entry. These bacterial proteins have entry specificity in leukemia cells, but not in normal PBMCs (FIG. 10, panel 1).

In addition to Azurin and Laz, Asparaginase and Arginine Deiminase (ADI) have been shown to be active against solid tumors including breast cancer, melanoma, renal cell carcinoma, hepatocellular carcinomas, etc., and ADI from Mycoplasma arginini (Ma-ADI) has also been reported to inhibit the proliferation of human leukemia cells. (Yamada T et al., Proc Natl Acad Sci USA 2002;99:14098-103; Punj V et al., Oncogene 23:2367-78 (2004); Ni Y et al., Cancer Lett. 261:1-11 (2008); Fialho A M, Chakrabarty A M, Anticancer Drug Discov 2:224-34 (2007); Ensor C M et al., Cancer Res 62:5443-50 (2002); Yoon C Y et al., Int. J Cancer 120:897-905 (2007); Gong H et al., Leukemia 14:826-9 (2000)). The catalytic triad in Ma-ADI is Cys 398-His 269-Glu213. Apart from a single report of Ma-ADI's effect on leukemia cells, however, previously little was known about the role of bacterial proteins, such as ADI, in possible therapeutic applications against leukemia.

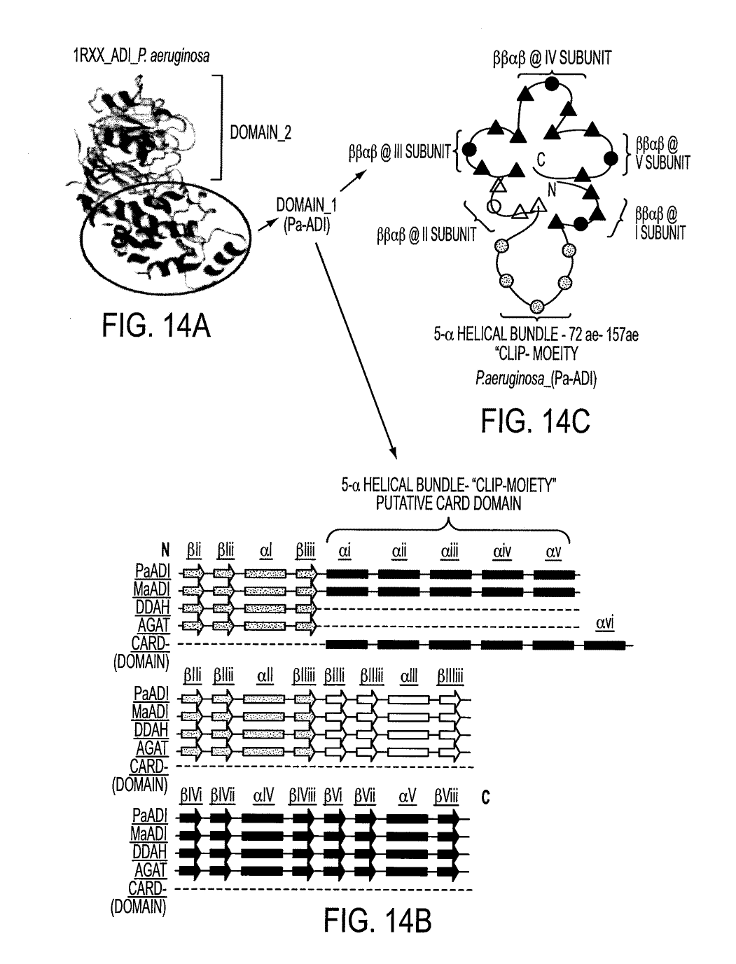

Pseudomonas aeruginosa also produces ADI (Pa-ADI), which has a catalytic triad at Cys406-His278-Asp166, and which has primary sequence homology with Ma-ADI. Use of a Secondary Structure Matching (SSM) program revealed a caspase recruitment domain (“CARD”)-like domain in the N-terminal region of Pa-ADI (Protein Data Bank, PDB ID code 1RXX), which has discernible structural homology to pro-domain of caspase-9 bearing a CARD domain (PDB ID code 3YGS). As shown in FIG. 14D, among the members of the guanidine group superfamily, ADI is unique in harboring the CARD domain. No CARD like domain in bacterial proteins is known other than a CARD-like domain in M. arginini ADI (PDB ID code 1LXY), discussed above. Surprisingly, as disclosed herein, the CARD domain in Pa-ADI (Pa-CARD) has strong anticancer activity. In short, the CARD domain polypeptide shows significant anticancer activity while exhibiting little cyotoxicity towards normal cells.

The mammalian CARD-containing proteins are normally composed of approximately 95 amino acid residues and have sequence similarity to death domains (DDs) and death effector domains (DEDs). The crystallographic structure of CARD suggests that it consists of about 6 tightly packed alpha-helices surrounding a hydrophobic pocket. Similarly, Pa-CARD consists of 85 amino acid residues forming the 5 packed alpha-helices which take the form of a “clip-on-fan” moiety (FIGS. 14B and 14C). Since some mammalian CARD domains promote cancer growth, an interesting possibility is that the bacterial CARD contributes to the interference in mammalian CARD activity, thereby contributing to the anticancer activity of the Pa-ADI containing the putative CARD-like domain. It is contemplated that the Pa-CARD domain may have been recruited from mammalian CARD-carrying proteins, and that Pa-CARD may exert its cytotoxic effects in cancer cells, at least in part, through protein-protein interactions with mammalian CARD proteins that are known to be hyperexpressed in cancer cells. It is further contemplated that this interaction may be a reason for the action of ADI in the modulation of cancer-growth.

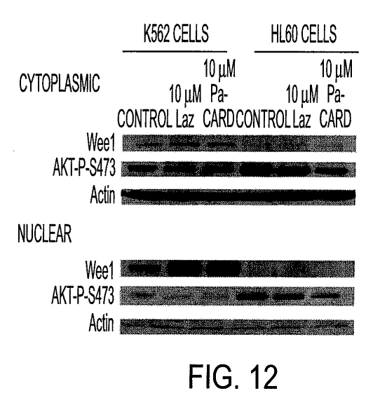

Pa-CARD (SEQ ID NO: 27), demonstrates surprising cytotoxic activity against leukemia cells and ovarian cancer cells. In particular, the Pa-CARD polypeptide has the ability to inhibit leukemia cell proliferation. In the leukemia cell lines HL60 and K562, the anticancer activity of Laz and Pa-CARD is mediated through cell cycle arrest at the G2/M phase involving the Wee1 protein stabilization and the depletion of phosphorylated AKT-Ser-473, the active form of a serine/threonine kinase that is often dysregulated in many cancer types. See Examples 11 and 12 and FIG. 12. Specifically, Pa-CARD and Laz inhibit cell cycle in leukemia cells through upregulation of the Wee1 protein in K562 cells and downregulation of active AKT P-Ser473 in HL60 cells (FIG. 12).

Both Wee1 and phosphorylated AKT-Ser473 (AKT-P-Ser473) are known to be involved in cell cycle arrest at the G2/M phase, which in turn is known to lead to cell death. For example, activation of the mitosis-promoting kinase, CDC2, also known as CDK1, is required for the transition from G2 to M phase in eukaryotic cells where phosphorylation of CDC2 on Thr-14 and Tyr-15 residues is important. Inhibitory Tyr-15 phosphorylation is mediated by the Wee1 protein kinase and therefore its enhanced levels mediate such inhibition of transition to the M phase. Viral proteins are known to mimic the Laz/Pa-CARD-mediated Wee protein kinase level elevation. The human papillomavirus type 1 (HPV-1) E4 protein inhibits G2 to M transition of the cell cycle through formation of inactive cyclin B1-CDK1 complexes through inhibitory Tyr-15 phosphorylation catalyzed by elevated levels of Wee1. Thus, overexpression of Wee1 enhances G2 cell cycle arrest while depletion of Wee1 by small interfering RNA (siRNA) alleviates the E4-induced G2 block. Similarly, AKT (protein kinase B), the human homologue of the viral oncogene v-akt, plays a role in the phosphatidyl inositol 3-kinase (PI3K) pathway. Phosphorylation in the Ser-473 residue in AKT-1 by PDK2 is important for cell cycle progression while a reduction in phosphorylated Akt Ser-473 (Akt-P-5473) levels enhances G2/M arrest, as evidenced by both Laz and Pa-CARD in HL60 cells.

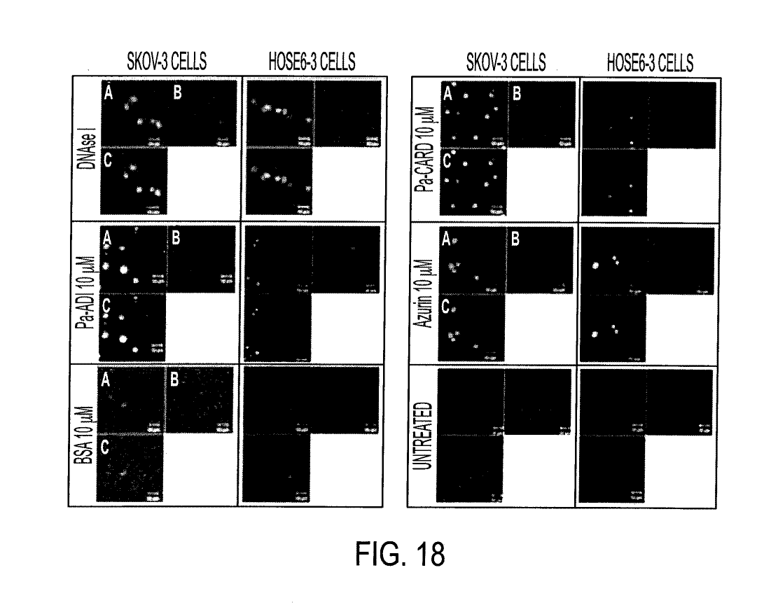

Pa-CARD has an effect on SKOV-3 (ovarian cancer) cells via the transcriptional stimulation of the gene encoding GM-CSF. Both GM-CSF and IL-12, whose gene is stimulated by about three fold, are known to be potent inducers of anti-tumor immunity in many tumor models, triggering infiltration of granulocytes, macrophages and dendritic cells at the tumor site, thereby greatly enhancing tumor antigen presentation. It thus appears that Pa-CARD-induced apoptosis (FIG. 18) may significantly add to the anti-tumor response of any GM-CSF hyperproduced in presence of Pa-CARD. While the gene profile expression showed stimulation of many cytokine genes, the gene for the chemokine CCL2 (C-C motif Ligand 2), also known as Monocyte Chemoattractant Protein-1 (MCP-1), showed significant (17 fold) repression (Table 3). CCL2 has often been shown to promote malignant transformation in breast cancer by increasing monocyte recruitment and deleterious Tumor-Associated Macrophages (TAM) in the tumor. CCL2 is also known to promote angiogenesis and metastasis presumably via MCPIP, a novel transcription factor, that activates transcription of the cadherin genes cdh12 and cdh19. Thus a reduction in CCL2 level by Pa-CARD may allow tumor regression by interfering in angiogenesis and tumor growth progression.

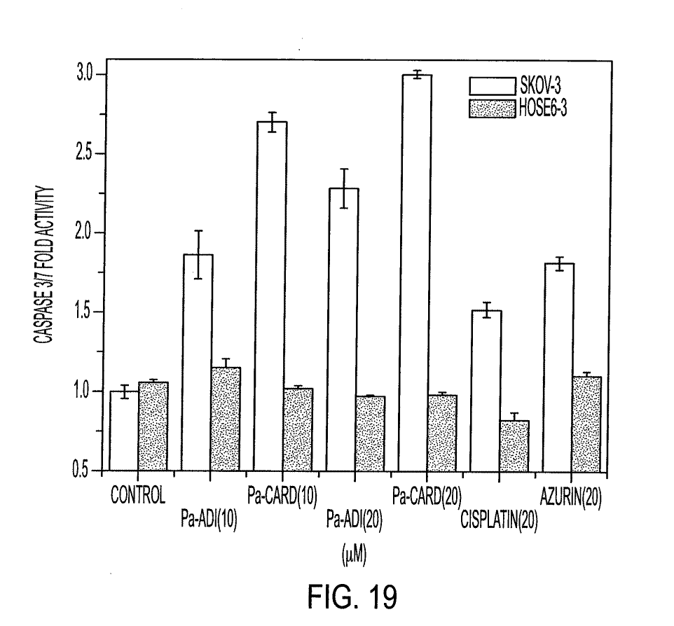

In summary, the Examples and disclosure herein demonstrate that bacterial proteins such as Azurin, Laz, and Pa-CARD can enter into leukemia cells and induce cell cycle arrest at the G2/M phase that appears to be triggered by modulation of cell signaling mediated by the CDC2-cyclin B repressive pathway and the cellular PI3K/AKT pathway. Furthermore, ADI can have anticancer activity in several ways, including via enzymatic depletion of arginine and through an N-terminal putative CARD-like domain without ADI activity (FIGS. 14-16). The Pa-cARD protein exhibits inhibitory role primarily to cancer cells and not to normal cells (FIG. 18). Furthermore, the anticancer activity may be mediated through caspase activity (FIG. 19), and can induce apoptosis in ovarian cancer cells.

### Compositions of the Invention

The invention provides for cytotoxic peptides and/or variants, derivatives, truncations, or structural equivalents of such peptides, alone or in combination with one or more other cytotoxic agents.

In some embodiments, the cytotoxic peptide is a cupredoxin or a truncation thereof. In some such embodiments, the cytotoxic peptide is an azurin or a truncation thereof. In other such embodiments, the cytotoxic peptide is Laz or a truncation thereof. In other embodiments, the cytotoxic peptide is the H.8 region of Laz. In some embodiments, the cytotoxic peptide comprises one or more of the amino acid sequences selected from the group consisting of SEQ ID NOS: 22-24.

In other embodiments, the cytotoxic peptide comprises a CARD domain. In some embodiments, the cytotoxic peptide is a CARD-carrying protein. In some embodiments, the CARD-carrying protein is derived from a bacteria. In another embodiment, the bacteria is P. aeruginosa. In one embodiment, the cytotoxic peptide is Pa-CARD. In another such embodiment, the cytotoxic peptide comprises SEQ ID NO: 27.

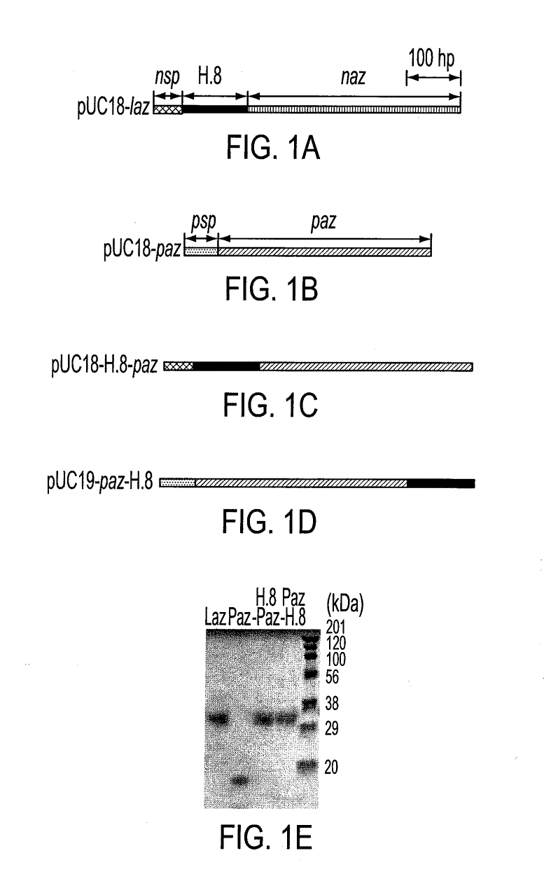

In some embodiments, the cytotoxic peptide is fused, using techniques well known in the art, with another peptide that confers upon it greater cytotoxic effects. In one such embodiment, the other peptide is the H.8 region of Laz. In another peptide, the other peptide has a sequence comprising SEQ ID NO: 24. In one embodiment, the cytotoxic peptide is the fusion protein Azu-H.8. In another embodiment, the cytotoxic peptide is the fusion protein H.8-Azu. FIG. 1.

The one or more other cytotoxic agents that may be administered in combination with the cytotoxic peptides described herein may be cancer treatment drugs including, but not limited to cisplatin, Gleevec®, Retinoic acid, 5′-aza-2′-deoxycytidine (“DAC”), and/or Retinoic acid in conjunction with arsenic trioxide. Cancer treatment drugs other than Gleevec®, Retinoic acid, DAC, and/or Retinoic acid in conjunction with arsenic trioxide include: a cell cycle control protein, such as p53; a cyclin-dependent kinase inhibitor, such as p16, p21 or p27; a suicide protein such as thymidine kinase or nitroreductase; a cytokine or other immunomodulatory protein such as interleukin 1, interleukin 2 or granulocyte-macrophage colony stimulating factor (GM-CSF); a toxin, such as Pseudomonas aeruginosa exotoxin A; 5-fluorouracil; Interferon α; Methotrexate; Tamoxifen; raloxifene; Vincrinstine; alkylating agents such as nitrogen mustards, alkyl sulfonates, nitrosoureas, ethylenimines, and triazenes; antimetabolites such as folate antagonists, purine analogues, and pyrimidine analogues; antibiotics such as anthracyclines, bleomycins, mitomycin, dactinomycin, and plicamycin; enzymes such as L-asparaginase; farnesyl-protein transferase inhibitors; 5alpha-reductase inhibitors; inhibitors of 17beta-hydroxysteroid dehydrogenase type 3; hormonal agents such as glucocorticoids, estrogens/antiestrogens, androgens/antiandrogens, progestins, and luteinizing hormone-releasing hormone antagonists, octreotide acetate; microtubule-disruptor agents, such as ecteinascidins or their analogs and derivatives; microtubule-stabilizing agents such as taxanes, for example, paclitaxel (Taxol™), docetaxel (Taxotere™), and their analogs, and epothilones, such as epothilones A-F and their analogs; plant-derived products, such as vinca alkaloids, epipodophyllotoxins, taxanes; and topiosomerase inhibitors; prenyl-protein transferase inhibitors; and miscellaneous agents such as hydroxyurea, procarbazine, mitotane, hexamethylmelamine, platinum coordination complexes such as cisplatin and carboplatin; and other agents used as anti-cancer and cytotoxic agents such as biological response modifiers, growth factors; immune modulators and monoclonal antibodies; mechlorethamine hydrochloride, cyclophosphamide, chlorambucil, melphalan, ifosfamide, busulfan, carmustin, lomustine, semustine, streptozocin, thiotepa, dacarbazine, methotrexate, thioguanine, mercaptopurine, fludarabine, pentastatin, cladribin, cytarabine, fluorouracil, doxorubicin hydrochloride, daunorubicin, idarubicin, bleomycin sulfate, mitomycin C, actinomycin D, safracins, saframycins, quinocarcins, discodermolides, vincristine, vinblastine, vinorelbine tartrate, etoposide, etoposide phosphate, teniposide, paclitaxel, estramustine, estramustine phosphate sodium, flutamide, buserelin, leuprolide, pteridines, diyneses, levamisole, aflacon, interferon, interleukins, aldesleukin, filgrastim, sargramostim, rituximab, BCG, tretinoin, irinotecan hydrochloride, betamethosone, gemcitabine hydrochloride, altretamine, and topoteca and any analogs or derivatives thereof.

Examples of other cytotoxic agents also include the following: epothilone derivatives as found in German Patent No. 4138042.8; WO 97/19086, WO 98/22461, WO 98/25929, WO 98/38192, WO 99/01124, WO 99/02224, WO 99/02514, WO 99/03848, WO 99/07692, WO 99/27890, WO 99/28324, WO 99/43653, WO 99/54330, WO 99/54318, WO 99/54319, WO 99/65913, WO 99/67252, WO 99/67253 and WO 00/00485; cyclin dependent kinase inhibitors as found in WO 99/24416 (see also U.S. Pat. No. 6,040,321); and prenyl-protein transferase inhibitors as found in WO 97/30992 and WO 98/54966; and agents such as those described generically and specifically in U.S. Pat. No. 6,011,029, the compounds of which can be employed together with any NHR modulators such as AR modulators, ER modulators, with LHRH modulators, especially in the treatment of cancer.

The invention further provides for cytotoxic peptides that are variants, derivatives, truncations or structural equivalents of cupredoxins and/or CARD-carrying proteins. In some embodiments, the cytotoxic peptide is isolated. In some embodiments, the cytotoxic peptide is substantially pure or pharmaceutical grade. In other embodiments, the cytotoxic peptide is in a composition that comprises, or consists essentially of, the cytotoxic peptide. In other embodiments, the cytotoxic peptide is in a composition that comprises both the cytotoxic peptide and at least one other cytotoxic agent. In another specific embodiment, the cytotoxic peptide is non-antigenic and does not raise an immune response in a mammal, and more specifically a human. In some embodiments, the cytotoxic peptide is less that a full-length cupredoxin or CARD-carrying protein, and retains some of the pharmacologic activities of the full length protein. Specifically, in some embodiments, the cytotoxic peptide may retain the ability to kill cancer cells, specifically leukemia cells.

Because of the high structural homology between the cupredoxins, it is contemplated that other cupredoxins will have the same cytotoxic properties as Azurin and Laz, specifically in regard to leukemia. In some embodiments, the cupredoxin is, but is not limited to, azurin, pseudoazurin, plastocyanin, rusticyanin, auracyanin, stellacyanin, cucumber basic protein or Laz. In particularly specific embodiments, the Azurin or Laz is derived from Pseudomonas aeruginosa, Alcaligenes faecalis, Achromobacter xylosoxidans ssp. denitrificans I, Bordetella bronchiseptica, Methylomonas sp., Neisseria meningitidis, Neisseria gonorrhea, Pseudomonas fluorescens, Pseudomonas chlororaphis, Xylella fastidiosa, Ulva pertussis or Vibrio parahaemolyticus. In a specific embodiment, the azurin is from Pseudomonas aeruginosa.

The invention provides cytotoxic peptides that are amino acid sequence variants of cupredoxins or CARD-carrying proteins which have amino acids replaced, deleted, or inserted as compared to the wild-type cupredoxin or CARD-carrying protein. Variants of the invention may be truncations of the wild-type protein. In some embodiments, the cytotoxic peptide of the invention comprises a region of a cupredoxin or CARD-carrying protein that is less that the full length wild-type polypeptide.

The cytotoxic peptides of the invention may also include peptides made with synthetic amino acids not naturally occurring. For example, non-naturally occurring amino acids may be integrated into the chemopreventive agent to extend or optimize the half-life of the composition in the bloodstream. Such chemopreventive agents include, but are not limited to, D,L-peptides (diastereomer), (for example Futaki et al., J. Biol. Chem. 276(8):5836-40 (2001); Papo et al., Cancer Res. 64(16):5779-86 (2004); Miller et al, Biochem. Pharmacol. 36(1):169-76, (1987).; peptides containing unusual amino acids (for example Lee et al., J. Pept. Res. 63(2):69-84 (2004)), olefin-containing non-natural amino acid followed by hydrocarbon stapling (for example Schafmeister et al., J. Am. Chem. Soc. 122:5891-5892 (2000); Walenski et al., Science 305:1466-1470 (2004)), and peptides comprising ε-(3,5-dinitrobenzoyl)-Lys residues.

In other embodiments, the cytotoxic peptide of the invention is a derivative of a cupredoxin or CARD-carrying protein. The derivatives of cupredoxin or CARD-carrying protein are chemical modifications of the peptide such that the peptide still retains some of its fundamental activities. For example, a “derivative” of azurin, Laz, or Pa-CARD can be a chemically modified protein that retains its ability to kill cancer cells, specifically leukemia and/or ovarian cancer cells. Another derivative may be one that has increased or optimized half-life in the bloodstream. The chemical modifications may include, but are not limited to, those disclosed in U.S. patent application Ser. No. 12/389,120, the disclosure of which is incorporated by reference in its entirety herein.

In some embodiments, the cytotoxic peptide may be modified using methods that include, but are not limited to, those which decrease the hydrolysis of the peptide, decrease the deamidation of the peptide, decrease the oxidation, decrease the immunogenicity and/or increase the structural stability of the peptide. It is contemplated that two or more of the modifications described or incorporated by reference herein may be combined in one modified cupredoxin derived peptide, as well as combinations of one or more modifications described herein with other modification to improve pharmacokinetic properties that are well known to those in the art. Many methods to design such variants and derivatives are well known in the art.

### Methods of Use

The invention provides methods of killing cancer cells, specifically leukemia and/or ovarian cancer cells, in mammals, specifically humans. The methods include contacting cancer cells with an isolated peptide that has a cytotoxic effect on cancer cells, or a variant, derivative, truncation, or structural equivalent thereof. The isolated peptide may be a cupredoxin or a CARD-carrying protein described herein. In one embodiment, the isolated peptide is an azurin. In another embodiment, the isolated peptide is Laz. In yet another embodiment, the isolated peptide is Pa-CARD. In further embodiments, the isolated peptide is a fusion protein comprising the H.8 region of Laz. In one such embodiment, the fusion protein is H.8-Azu. In another such embodiment, the fusion protein is Azu-H.8. The cytotoxic peptide may be administered to the cancer cells alone or in combination with another cytotoxic agent, as described herein, or in combination with the H.8 region of Laz. In some embodiments, the cytotoxic peptide is administered at or around the same time as the H.8 region of Laz. In other embodiments, the cytotoxic peptide is administered at or around the same time as a sequence comprising SEQ ID NO: 24.

The invention also provides methods of treating mammalian patients with cancer, or otherwise killing cancer cells in mammalian patients, by administering to the patient an isolated peptide that has a cytotoxic effect on cancer cells or a variant, derivative, truncation, or structural equivalent thereof. The isolated peptide may be a cupredoxin or a CARD-carrying protein described herein. In one embodiment, the isolated peptide is an azurin. In another embodiment, the isolated peptide is Laz. In yet another embodiment, the isolated peptide is Pa-CARD. In further embodiments, the isolated peptide is a fusion protein comprising the H.8 region of Laz. In one such embodiment, the fusion protein is H.8-Azu. In another such embodiment, the fusion protein is Azu-H.8. The cytotoxic peptide may be administered alone or in combination with another cytotoxic agent, as described herein, or in combination with the H.8 region of Laz. In some embodiments, the cytotoxic peptide is administered at or around the same time as the H.8 region of Laz. In other embodiments, the cytotoxic peptide is administered at or around the same time as a sequence comprising SEQ ID NO: 24.

The invention also provides methods of inducing the death of leukemia and/or ovarian cancer cells through cellular differentiation, by administering to leukemia and/or ovarian cancer cells one or more cytotoxic peptides. The cytotoxic peptide may be a cupredoxin or a CARD-carrying protein described herein. In one embodiment, the cytotoxic peptide is Azurin. In another embodiment, the cytotoxic peptide is Laz. In another embodiment, the cytotoxic peptide is the fusion protein H.8-Azu. In yet another embodiment, the cytotoxic peptide is the fusion protein Azu-H.8.

The invention also provides methods of selectively entering leukemia and/or ovarian cancer cells and having cytotoxic effects therein, by administering to leukemia and/or ovarian cancer cells one or more cytotoxic peptides. The cytotoxic peptide may be a cupredoxin or a CARD-carrying protein described herein. In one embodiment, the cytotoxic peptide is Azurin. In another embodiment the cytotoxic peptide is Laz. In yet another embodiment, the cytotoxic peptide is Pa-CARD. In further embodiments, the cytotoxic peptide is a fusion protein comprising the H.8 region of Laz. In one such embodiment, the fusion protein is H.8-Azu. In another such embodiment, the fusion protein is Azu-H.8.

The invention further includes methods of killing leukemia and/or ovarian cancer cells by causing cell cycle arrest at the G2/M phase, comprising administering one or more cytotoxic peptides. In a further embodiment, the cytotoxic peptide stabilizes the Wee1 protein and/or increases the levels of Wee1 protein in the cell, including but not limited to in the cytoplasm and/or nucleus of the cell. In yet another embodiment, the cytotoxic peptide depletes phosphorylated AKT-Ser-473. In yet another embodiment, the cytotoxic peptide both stabilizes/increases levels of the Wee1 protein and depletes phosphorylated AKT-Ser-473. In a further embodiment, the cytotoxic peptide is Azurin. In another embodiment, the cytotoxic peptide is Laz. In yet another embodiment, the cytotoxic peptide is Pa-CARD. In yet another embodiment, the cytotoxic peptide is one or more of the fusion proteins H.8-Azu and Azu-H.8.

### Nucleic Acids Coding for a Cytotoxic Peptide and Expression Vectors

In another aspect, the present invention provides nucleic acid molecules encoding the cytotoxic peptides and variants, derivatives, and/or structural equivalents thereof described herein. The nucleic acid molecule according to the invention can be prepared by a combination of known techniques in the art. The coding sequences used in these nucleic acids may be those found in the native genomic DNA encoding the particular peptide, or may be designed from known codons. These coding sequences may also be designed to take into account alternate codon usage and preferred codon usage of the organism in which the peptide is to be expressed. The nucleic acid sequences for the cytotoxic peptides may individually be prepared by chemical synthesis or cloning. The nucleic acid sequences may be then ligated together with ligase in order to give a nucleic acid molecule of interest.

Vectors, used to shuttle genetic material from organism to organism, can be divided into two general classes: cloning vectors are replicating plasmid or phage with regions that are essential for propagation in an appropriate host cell and into which foreign DNA can be inserted; the foreign DNA is replicated and propagated as if it were a component of the vector. An expression vector (such as a plasmid, yeast, or animal virus genome) is used to introduce foreign genetic material into a host cell or tissue in order to transcribe and translate the foreign DNA, such as the DNA of a cytotoxic peptide like Laz, Azurin, Pa-CARD, H.8-Azu, or Azu-H.8. In expression vectors, the introduced DNA is operably-linked to elements such as promoters that signal to the host cell to highly transcribe the inserted DNA. Some promoters are exceptionally useful, such as inducible promoters that control gene transcription in response to specific factors. Operably-linking a cytotoxic peptide and variants and derivatives thereof to an inducible promoter can control the expression of the cytotoxic peptide and variants and derivatives thereof in response to specific factors. Examples of classic inducible promoters include those that are responsive to alpha-interferon, heat shock, heavy metal ions, and steroids such as glucocorticoids (Kaufman, Methods Enzymol. 185:487-511(1990)) and tetracycline. Other desirable inducible promoters include those that are not endogenous to the cells in which the construct is being introduced, but are responsive in those cells when the induction agent is exogenously supplied. In general, useful expression vectors are often plasmids. However, other forms of expression vectors, such as viral vectors (e.g., replication defective retroviruses, adenoviruses, and adeno-associated viruses) are contemplated.

Vector choice is dictated by the organism or cells being used and the desired fate of the vector. In general, vectors comprise signal sequences, origins of replication, marker genes, polylinker sites, enhancer elements, promoters, and transcription termination sequences.

### Pharmaceutical Compositions Comprising a Cytotoxic Peptide

The invention also provides compositions comprising at least one cytotoxic peptide that is a cupredoxin or CARD-carrying protein, or variant, derivative, truncation, or structural equivalent of a cupredoxin or CARD-carrying protein, specifically in a pharmaceutical composition, alone or in combination with at least one other cytotoxic agent. In specific embodiments, the pharmaceutical composition is designed for a particular mode of administration, for example, but not limited to, oral, intraperitoneal, or intravenous. Such compositions may be hydrated in water, or may be dried (such as by lyophilization) for later hydration. Such compositions may be in solvents other than water, such as but not limited to, alcohol.

Pharmaceutical compositions of the invention containing a cytotoxic peptide can be manufactured in any conventional manner, e.g., by conventional mixing, dissolving, granulating, dragee-making, emulsifying, encapsulating, entrapping, or lyophilizing processes. The cytotoxic peptide can be readily combined with a pharmaceutically acceptable carrier well-known in the art. Such carriers enable the preparation to be formulated as a tablet, pill, dragee, capsule, liquid, gel, syrup, slurry, suspension, and the like. Suitable excipients may also include, for example, fillers and cellulose preparations. Other excipients can include, for example, flavoring agents, coloring agents, detackifiers, thickeners, and other acceptable additives, adjuvants, or binders.

In various embodiments, the composition includes carriers and excipients (including but not limited to buffers, carbohydrates, mannitol, proteins, polypeptides or amino acids such as glycine, antioxidants, bacteriostats, chelating agents, suspending agents, thickening agents and/or preservatives), water, oils, saline solutions, aqueous dextrose and glycerol solutions, other pharmaceutically acceptable auxiliary substances as required to approximate physiological conditions, such as buffering agents, tonicity adjusting agents, wetting agents and the like. It will be recognized that, while any suitable carrier known to those of ordinary skill in the art may be employed to administer the compositions of this invention, the type of carrier will vary depending on the mode of administration. Compounds may also be encapsulated within liposomes using well-known technology. Biodegradable microspheres may also be employed as carriers for the compositions of this invention. Suitable biodegradable microspheres are shown, for example, in U.S. Pat. Nos. 4,897,268, 5,075,109, 5,928,647, 5,811,128, 5,820,883, 5,853,763, 5,814,344 and 5,942,252. “Compounds” as used herein, include the peptides, amino acid sequences, cargo compounds and complexes, and nucleic acids of the present invention.

Intravenous fluids for use in preparing pharmaceutical compositions to administer the cytotoxic peptides and nucleic acids disclosed herein may be composed of crystalloids or colloids. Crystalloids as used herein are aqueous solutions of mineral salts or other water-soluble molecules. Colloids as used herein contain larger insoluble molecules, such as gelatin. Intravenous fluids may be sterile.

Crystalloid fluids that may be used for intravenous administration include but are not limited to, normal saline (a solution of sodium chloride at 0.9% concentration), Ringer's lactate or Ringer's solution, and a solution of 5% dextrose in water sometimes called D5W, as described in Table 1.

TABLE 1
Composition of Common Crystalloid Solutions

| Solution | Other Name | [Na⁺] | [Cl⁻] | [Glucose] |
|---|---|---|---|---|
| D5W | 5% Dextrose | 0 | 0 | 252 |
| ⅔ & ⅓ | 3.3% Dextrose/ | 51 | 51 | 168 |
| | 0.3% saline | | | |
| Half-normal | 0.45% NaCl | 77 | 77 | 0 |
| saline | | | | |
| Normal saline | 0.9% NaCl | 154 | 154 | 0 |
| Ringer's | Ringer's | 130 | 109 | 0 |
| lactate* | solution | | | |

*Ringer's lactate also has 28 mmol/L lactate, 4 mmol/L K⁺ and 3 mmol/L Ca²⁺.

The half-life in the bloodstream of the compositions of the invention can be extended or optimized by several methods well known to those in the art, including but not limited to, circularized peptides (Monk et al., BioDrugs 19(4):261-78, (2005); DeFreest et al., J. Pept. Res. 63(5):409-19 (2004)), D,L-peptides (diastereomer), (Futaki et al., J. Biol. Chem. February 23; 276(8):5836-40 (2001); Papo et al., Cancer Res. 64(16):5779-86 (2004); Miller et al., Biochem. Pharmacol. 36(1):169-76, (1987)); peptides containing unusual amino acids (Lee et al., J. Pept. Res. 63(2):69-84 (2004)), and N- and C-terminal modifications (Labrie et al., Clin. Invest. Med. 13(5):275-8, (1990)). Of particular interest are d-isomerization (substitution) and modification of peptide stability via D-substitution or L-amino acid substitution.

When administration is by injection, composition may be formulated in aqueous solutions, preferably in physiologically compatible buffers such as Hanks solution, Ringer's solution, or physiological saline buffer. The solution may contain formulatory agents such as suspending, stabilizing and/or dispersing agents. Alternatively, the composition may be in powder form for constitution with a suitable vehicle, e.g., sterile pyrogen-free water, before use.

When administration is by inhalation, the composition may be delivered in the form of an aerosol spray from pressurized packs or a nebulizer with the use of a suitable propellant, e.g., dichlorodifluoromethane, trichlorofluoromethane, carbon dioxide or other suitable gas. In the case of a pressurized aerosol, the dosage unit may be determined by providing a valve to deliver a metered amount. Capsules and cartridges of, e.g., gelatin for use in an inhaler or insufflator may be formulated containing a powder mix of the proteins and a suitable powder base such as lactose or starch.

When administration is by topical administration, the composition may be formulated as solutions, gels, ointments, creams, suspensions, and the like, as are well known in the art. In some embodiments, administration is by means of a transdermal patch. When administration is by suppository (e.g., rectal or vaginal), composition may also be formulated in compositions containing conventional suppository bases. When administration is oral, the composition can be readily formulated in combination with pharmaceutically acceptable carriers well known in the art. A solid carrier, such as mannitol, lactose, magnesium stearate, and the like may be employed; such carriers enable the chemotaxin to be formulated as tablets, pills, dragees, capsules, liquids, gels, syrups, slurries, suspensions and the like, for oral ingestion by a subject to be treated. For oral solid formulations such as, for example, powders, capsules and tablets, suitable excipients include fillers such as sugars, cellulose preparation, granulating agents, and binding agents.

Other convenient carriers, as well-known in the art, also include multivalent carriers, such as bacterial capsular polysaccharide, a dextran or a genetically engineered vector. In addition, sustained-release formulations that include the composition allow for the release of the composition over extended periods of time, such that without the sustained release formulation, composition would be cleared from a subject's system, and/or degraded by, for example, proteases and simple hydrolysis before eliciting or enhancing an therapeutic effect.

### Kits Comprising a Cytotoxic Peptide

In another aspect, the invention provides kits containing one or more of the following in a package or container: (1) a reagent comprising one or more cytotoxic peptides described herein; (2) a reagent containing a pharmaceutically acceptable adjuvant or excipient; (3) a vehicle for administration, such as a syringe; (4) instructions for administration. Embodiments in which two or more of components (1)-(4) are found in the same container are also contemplated. In other embodiments, the kit components may also include one or more additional cytotoxic agents. In other embodiments, the reagents are formulated for intravenous administration, and/or the vehicle of administration is appropriate for intravenous administration.

When a kit is supplied, the different components of the composition may be packaged in separate containers and admixed immediately before use. Such packaging of the components separately may permit long-term storage without losing the active components' functions.

The reagents included in the kit can be supplied in containers of any sort such that the life of the different components are preserved and are not adsorbed or altered by the materials of the container. For example, sealed glass ampules may contain lyophilized polypeptide or polynucleotide, or buffers that have been packaged under a neutral, non-reacting gas, such as nitrogen. Ampules may consist of any suitable material, such as glass, organic polymers, such as polycarbonate, polystyrene, etc., ceramic, metal or any other material typically employed to hold similar reagents. Other examples of suitable containers include simple bottles that may be fabricated from similar substances as ampules, and envelopes, that may comprise foil-lined interiors, such as aluminum or an alloy. Other containers include test tubes, vials, flasks, bottles, syringes, or the like. Containers may have a sterile access port, such as a bottle having a stopper that can be pierced by a hypodermic injection needle. Other containers may have two compartments that are separated by a readily removable membrane that upon removal permits the components to be mixed. Removable membranes may be glass, plastic, rubber, etc.

Kits may also be supplied with instructional materials. Instructions may be printed on paper or other substrate, and/or may be supplied as an electronic-readable medium, such as a floppy disc, CD-ROM, DVD-ROM, Zip disc, videotape, audiotape, flash memory device, etc. Detailed instructions may not be physically associated with the kit; instead, a user may be directed to an internet web site specified by the manufacturer or distributor of the kit, or supplied as electronic mail.

A more complete understanding of the present invention can be obtained by reference to the following specific Examples. The Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitations. Modifications and variations of the invention as hereinbefore set forth can be made without departing from the spirit and scope thereof, and, therefore, only such limitations should be imposed as are indicated by the appended embodiments.

### Examples

### Example 1

### Cloning and Expression of the Laz and H.8-Azurin Fusion Genes

The laz gene from Neisseria gonorrhoeae was cloned (FIG. 1A) based on its known sequence (SEQ ID NO: 1). The P. aeruginosa azurin gene (SEQ ID NO: 2), termed paz (FIG. 1B), and the sequence of the H.8 epitope of laz from N. gonnerrhoeae (SEQ ID NO: 3), were used to clone in frame the H.8 epitope gene in the 5′-end of paz to produce H.8-paz (FIG. 1C) or in the 3′-end of paz to generate paz-H.8 (FIG. 1D), as described below.

Cell Lines and Reagents. Human cancer cells, bacterial strains and plasmids are listed in Table 2. The human breast cancer MCF-7 cells and brain tumor LN-229 cells are from the stock culture collection of the Department of Surgical Oncology, University of Illinois at Chicago (UIC). The cells were cultured in MEM with Eagle's salt containing 2 mM L-glutamine, 0.1 mM MEM essential amino acids and supplemented with 10% heat-inactivated fetal bovine serum, 100 units/ml penicillin and 100 μg/ml streptomycin. All cells were grown at 37° C. in 5% CO₂. (Yamada, et al., Proc. Natl. Acad. Sci. USA 99:14098-14103 (2002); Punj, et al., Oncogene 23:2367-2378 (2004)).

TABLE 2
Cancer cells, bacterial strains and genetic constructs

| Cells/strains/ | | |
|---|---|---|
| plasmids | Relevant characteristics* | Reference |
| LN-229 | Human brain glioblastoma | Ishii, et al., Brain Pathol. |
| | | 9: 469-479 (1999) |
| MCF-7 | Human breast adenocarcinoma | Soule, et al., J. Natl. |
| | | Cancer. Inst. 51: 1409-1416 |
| | | (1973); Punj, et al., |
| | | Oncogene 23: 2367-2378 |
| | | (2004) |
| P. aeruginosa | Prototroph, FP-(sex factor minus) | Holloway, et al., |
| PAO1 | | Microbiol. Rev. 43: 73-102 |
| | | (1979) |
| E. coli JM109 | Cloning and azurin expression strain | Yanisch-Perron, et al., |
| | | Gene 33: 103-119 (1985) |
| E. coli BL21 | GST expression strain | Novagen |
| (DE3) | | |
| N. gonorrhoeae | Prototroph used for DNA isolation | American Type Culture |
| F62 | | Collection |
| pUC18 | General cloning vector, Ap^{r} | Yanisch-Perron, et al., id. |
| pUC19 | General cloning vector, Ap^{r} | Yanisch-Perron, et al., id. |
| pUC18-laz | A 1 kb PCR fragment from genomic | Herein |
| | DNA of N. gonorrhoeae F62 cloned | |
| | into pUC18 | |
| pUC19-paz | A 0.55 kb PCR fragment from P. aeruginosa | Yamada, et al., Proc. Natl. |
| | PAO1 cloned into HindIII | Acad. Sci. USA 99: 14098-14103 |
| | and PstI digested pUC19, Ap^{r} | (2002); Yamada, et |
| | | al., Proc. Natl. Acad. Sci. |
| | | USA 101: 4770-4775 |
| | | (2004) |
| pUC18-H.8- | Fusion plasmid encoding H.8 from N. gonorrhoeae | Herein |
| paz | and azurin from P. aeruginosa | |
| | PAO1, Ap^{r} | |
| pUC19-paz- | Fusion plasmid encoding azurin from | Herein |
| H.8 | P. aeruginosa PAO1 and H.8 from N. gonorrhoeae, | |
| | Ap^{r} | |
| pGEX-5X-3 | GST gene fusion vectors, Ap^{r} | Amersham |
| pET29a | E. coli expression vector, Km^{r} | Novagen |
| pET29a-gst | pET29a derivative containing the gst | Herein |
| | gene, Km^{r} | |
| pET29a-H.8- | pET29a derivative containing H.8-gst | Herein |
| gst | gene, Km^{r} | |
| pGEX-5X-3- | pGEX-5X-3 derivative containing H.8- | Herein |
| H.8 | encoding region, Ap^{r} | |
| pET29a-gst- | pET29a derivative containing gst-H.8 | Herein |
| H.8 | gene, Km^{r} | |

*Ap, ampicillin; Km, kanamycin; GST, Glutathione S-transferase.

Cloning and Expression of the paz and laz Genes. The cloning and hyperexpression of the azurin gene has been described. (Yamada, et al., Proc. Natl. Acad. Sci. USA 99:14098-14103 (2002); Punj, et al., Oncogene 23:2367-2378 (2004)) The Laz-encoding gene (laz) of Neisseria gonorrhoeae was amplified by PCR with genomic DNA of N. gonorrhoeae strain F62 as template DNA. The forward and reverse primers used were 5′-CCGGAATTCCGGCAGGGATGTTGTAAATATCCG-3′ (SEQ ID NO: 4) and 5′-GGGGTACCGCCGTGGCAGGCATACAGCATTTCAATCGG-3′ (SEQ ID NO: 5) where the additionally introduced restriction sites of EcoRI and KpnI sites are underlined respectively. The amplified DNA fragment of 1.0 kb, digested with EcoRI and KpnI, was inserted into the corresponding sites of pUC18 vector (Yanisch-Perron, et al., Gene 33:103-119 (1985)) so that the laz gene was placed downstream of the lac promoter to yield an expression plasmid pUC18-laz (Table 2, FIG. 1).

The plasmids expressing fusion H.8 of N. gonorrhoeae Laz and azurin of P. aeruginosa (Paz) were constructed by PCR with pUC19-paz and pUC18-laz as templates. For H.8-Paz fusion, a 3.1 kb fragment was amplified with pUC18-laz as a template and primers, 5′-(phosphorylated)GGCAGCAGGGGCTTCGGCAGCATCTGC-3′ (SEQ ID NO: 6) and 5′-CTGCAGGTCGACTCTAGAGGATCCCG-3′ (SEQ ID NO: 7) where a SalI site is underlined. A PCR amplified a 0.4 kb fragment was obtained from pUC 19-paz as a template and primers, 5′-(phosphorylated)GCCGAGTGCTCGGTGGACATCCAGG-3′ (SEQ ID NO: 8) and 5′-TACTCGAGTCACTTCAGGGTCAGGGTG-3′ (SEQ ID NO: 9) where a XhoI site is underlined. A SalI digested PCR fragment from pUC18-laz and XhoI digested PCR fragment from pUC19-paz were cloned to yield an expression plasmid pUC18-H.8-paz (Table 2, FIG. 1).

For Paz-H.8 fusion, a 3.3 kb fragment was amplified with pUC19-paz as a template and primers, 5′-CTTCAGGGTCAGGGTGCCCTTCATC-3′ (SEQ ID NO: 10) and 5′-CTGCAGGTCGACTCTAGAGGATCCCG-3′ (SEQ ID NO: 11) where a BamHI site is underlined. A 0.13 kb fragment was amplified with pUC18-laz as a template and primers, 5′-(phosphorylated)TGCTCTCAAGAACCTGCCGCGCCTGC-3′ (SEQ ID NO: 12) and 5′-TAGGATCCTTAGGCAGCAGGGGCTTCGGCAGCATCTGC-3′ (SEQ ID NO: 13) where a BamHI site is underlined and the additionally introduced TTA, corresponding to the bacterial gene stop codon, is italicized. Two BamHI digested PCR fragments were cloned to yield an expression plasmid pUC19-paz-H.8 (Table 2).

E. coli JM109 was used as a host strain for expression of azurin and its derivative genes. Recombinant E. coli strains were cultivated in 2× YT medium containing 100 μg/ml ampicillin, 0.1 mM IPTG and 0.5 mM CuSO₄ for 16 h at 37° C. to produce the azurin proteins.

Plasmid Construction for Fusion GST Proteins. The glutathione S-transferase (GST)-encoding gene was amplified by PCR with pGEX-5X-3 (GE Healthcare Bio-Sciences Corp., Piscataway, N.J.) as template DNA. The forward and reverse primers used were 5′-CGAGCTCATGTCCCCTATACTAGGTTATTGG-3′ (SEQ ID NO: 14) and 5′-CCCAAGCTTTCAGGGGATCCCACGACCTTCGATCAGATCC-3′ (SEQ ID NO: 15) where the additionally introduced restriction sites of SacI and HindIII are underlined and the additionally introduced TCA, corresponding bacterial gene stop codon, is italicized respectively. The amplified DNA fragment of 1.0 kb, digested with SacI and HindIII, was inserted into the corresponding sites of pET29a vector to yield an expression plasmid pET29a-gst (Table 2).

For H.8-GST fusion, the signal peptide and H.8-encoding region of laz was amplified by PCR with pUC18-laz as template DNA. The forward and reverse primers used were 5′-GGAATTCATATGAAAGCTTATCTGGC-3′ (SEQ ID NO: 16) and 5′-CCGGAATTCGGCAGCAGGGGCTTCGGC-3′ (SEQ ID NO: 17) where the additionally introduced restriction sites of NdeI and EcoRI sites are underlined respectively. The amplified DNA fragment of 0.14 kb, digested with NdeI and EcoRI, was inserted into the corresponding sites of pET29a-gst vector to yield an expression plasmid pET29a-H.8-gst (Table 2).

For GST-H.8 fusion, the H.8-encoding region was amplified by PCR with pUC18-laz as template DNA. The forward and reverse primers used were 5′-CGGGATCCCCTGCTCTCAAGAACCTGCCGCGCC-3′ (SEQ ID NO: 18) and 5′-CGGAATTCTTAGGCAGCAGGGGCTTCGGCAGCATCTGCAGG-3′ (SEQ ID NO: 19) where the additionally introduced restriction sites of BamHI and EcoRI are underlined and the introduced bacterial gene stop codon TTA is italicized. The amplified DNA fragment of 0.14 kb, digested with BamHI and EcoRI, was inserted into the corresponding sites of pGEX-5X-3 vector to yield a pGEX-5X-3-H.8. The GST-H.8 fusion region was then amplified by PCR with pGEX-5X-3-H.8 as a template DNA. The forward and reverse primers used were 5′-CGAGCTCATGTCCCCTATACTAGGTTATTGG-3′ (SEQ ID NO: 20) and 5′-CCGCTCGAGTCAGGCAGCAGGGGCTTCGGCAG-3′ (SEQ ID NO: 21) where the additionally introduced restriction sites of SacI and XhoI sites are underlined and the bacterial gene stop codon TCA is italicized. The amplified DNA fragment of 1.1 kb, digested with SacI and XhoI, was inserted into the corresponding sites of pET29a vector to yield an expression plasmid pET29a-gst-H.8 (Table 2).

E. coli BL21 (DE3) was used as a host strain for expression of the gst and its fusions derivatives. When E. coli strains harboring these plasmids were grown in presence of IPTG, cells lysed and the proteins purified as described for azurin (Yamada, et al., Proc. Natl. Acad. Sci. USA 99:14098-14103 (2002); Punj, et al., Oncogene 23:2367-2378 (2004); Yamada, et al., Cell. Microbiol. 7:1418-1431 (2005)), the various azurin derivatives migrated on SDS-PAGE as single components (FIG. 1E), although the H.8 containing proteins (about 17 kDa) showed anomalous migrations, as noted before (Cannon et al., id.; Fisette et al., id.).

### Example 2

### H.8 Enhances the Cytotoxicity of P. aeruginosa Azurin Towards Glioblastoma Cells but not Breast Cancer Cells

The preferential entry of Paz towards cancer cells (Yamada, et al., Cell. Microbiol. 7:1418-1431 (2005)) and its cytotoxicity, both in vitro and in vivo towards human melanoma (Yamada, et al., Proc. Natl. Acad. Sci. USA 99:14098-14103 (2002)) and breast cancer (Punj, et al., Oncogene 23:2367-2378 (2004)), have been reported. See also U.S. patent application Ser. No. 12/338,480, the disclosure of which is incorporated by reference in its entirety herein. However, no effect of Paz or Laz towards brain tumors such as glioblastomas is known. Here the effect of Paz, Laz, H.8-Paz (H.8 epitope in the N-terminal of Paz) and Paz-H.8 (H.8 epitope in the C-terminal of Paz) on both glioblastoma (LN-229 cell line) and breast cancer (MCF-7 cell line) cells was studied.

Preparations of Proteins. Azurin (Paz) of P. aeruginosa, Laz of N. gonorrhoeae, Paz-H.8 and H.8-Paz were purified as described previously. (Yamada, et al., Proc. Natl. Acad. Sci. USA 99:14098-14103 (2002); Punj, et al., Oncogene 23:2367-2378 (2004); Yamada, et al., Cell. Microbiol. 7:1418-1431 (2005)) All recombinant GST fusion derivatives were purified as described before. (Yamada, et al., Cell. Microbiol. 7:1418-1431 (2005)) A chemically-synthesized 39-amino acid H.8 peptide was purchased.

Cytotoxicity Assay. The 3-(4,5-dimethylthiazol-2-yl-2,5-diphenyl)tetrazolium bromide (MTT) assay was performed to determine the cytotoxicity toward cancer cells. Cells (5×10³ per well) were seeded into 96-well culture plates in 100:1 of the medium at 37° C. with 5% CO₂. After overnight incubation, the supernatant was removed and fresh media containing proteins at various concentrations as specified were added to the attached cells. These cells were incubated for various time periods as specified before the number of live cells was determined by MTT assay by adding 10 μl of 5 mg/ml MTT (Sigma-Aldrich, St. Louis, Mo.) solution to the culture and incubating for 2 h at 37° C. MTT reaction was terminated by adding 100 μl of 40 mM HCl in isopropanol. The MTT formazan formed was measured spectrophotometrically according to the method described by Mosmann (J. Immunol. Methods 65:55-63 (1983)).

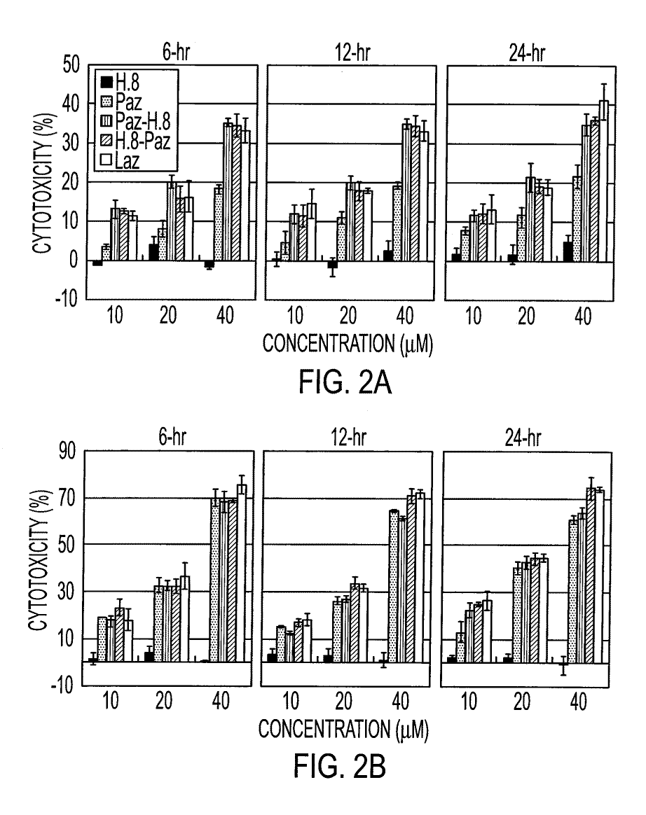

The synthetic H.8 peptide had very little cytotoxicity towards either glioblastoma LN-229 (FIG. 2A) or breast cancer MCF-7 (FIG. 2B) cells. The effect of azurin (Paz) was dose dependent, albeit low, in glioblastoma (FIG. 2A) but not in breast cancer (FIG. 2B) cells with increasing cytotoxicity as the azurin concentration was raised from 10 μM to 40 μM. The cytotoxicity increased only marginally beyond a 6 h incubation period. Most notable was the difference in the cytotoxicity of Paz, Paz-H.8, H.8-Paz and Laz in glioblastoma and breast cancer cells. While Paz, Paz-H.8, H.8-Paz and Laz had essentially identical cytotoxicities at all doses in MCF-7 cells for different periods of incubation (FIG. 2B), Paz had much lower cytotoxicity than Paz-H.8, H.8-Paz or Laz for glioblastoma cells, particularly at shorter periods of incubation (6 h). Thus the H.8 moiety, while itself lacking cytotoxicity, appeared to enhance the cytotoxicity of Paz, but only towards glioblastoma and not towards breast cancer cells.

### Example 3

### H.8 Epitope Present in Paz or Laz Facilitates the Uptake of Azurin in Glioblastoma Cells

The enhanced cytotoxicity of Paz-H.8, H.8-Paz and Laz towards glioblastoma cells as compared to Paz, raised the question if the H.8 moiety somehow facilitated the uptake of azurin in glioblastoma cells. Alexa fluor® 568-labeled red fluorescent proteins (Invitrogen-Molecular Probes Corp., Carlsbad, Calif.) were used to determine the internalization of these proteins inside glioblastoma and breast cancer cells. This technique was previously used to demonstrate the internalization of azurin in MCF-7 cells (Punj, et al., Oncogene 23:2367-2378 (2004); Yamada, et al., Cell. Microbiol. 7:1418-1431 (2005)).

Confocal Microscopy. For preparation of microscopic samples, cells were cultured on coverslips overnight at 37° C. under 5% CO₂. Pre-warmed 37° C. fresh media were mixed with red fluorescent-labeled (Alexa fluor® 568) azurin or GST fusion derivatives, and incubated with the cells for indicated times. The cells were washed with PBS, and fixed with methanol at −20° C. for 5 min. After washing with PBS thrice and the addition of mounting media containing 1.5 mg/ml 4,6-diamidino-2-phenylindole (DAPI) for staining nuclei (VECTASHIELD®, Vector Laboratories, Burlingame, Calif.), images were taken by using a Carl Zeiss LSM510 laser scanning confocal microscope. (Yamada, et al., Cell. Microbiol. 7:1418-1431 (2005)).

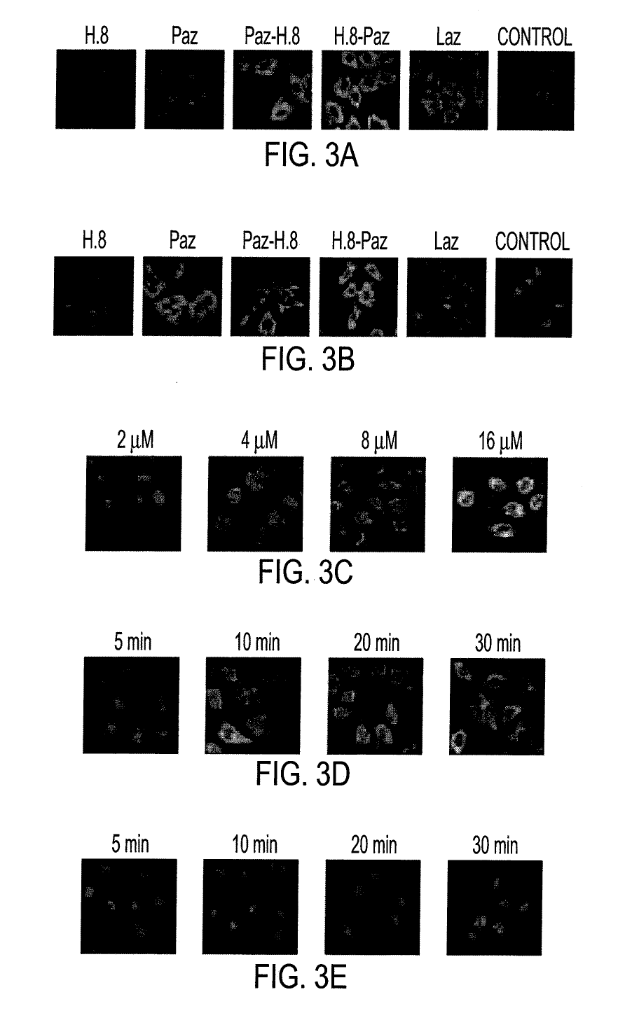

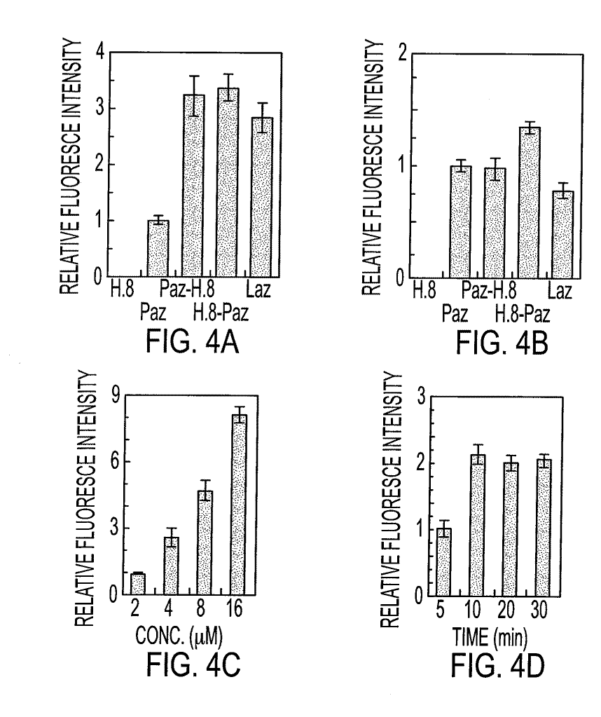

Azurin (Paz) was internalized with a reduced efficiency than Paz-H.8, H.8-Paz and Laz, demonstrating a barrier for Paz entry in glioblastoma LN-229 cells (FIGS. 3A and 4A). In contrast, Paz was efficiently internalized in breast cancer MCF-7 cells as previously reported, with an equal or somewhat higher efficiency than Paz-H.8, H.8-Paz or Laz (FIGS. 3B and 4B). (Punj, et al., Oncogene 23:2367-2378 (2004); Yamada, et al., Cell. Microbiol. 7:1418-1431 (2005)) A dose dependency of Laz entry in LN-229 cells demonstrated an optimum concentration of about 16 μM during a 30 min incubation period at 37° C. (FIGS. 3C and 4C) beyond which there was no further enhancement (data not shown). At 10 μM concentration, while the bulk of Laz was internalized in LN-229 cells in about 10 to 20 min (FIGS. 3D and 4D), the internalization of Paz was minimal under such conditions (FIG. 3E), suggesting that Paz internalization was inherently inefficient in LN-229 cells. The significant internalization of Paz-H.8 and H.8-Paz, similar to Laz but in contrast to Paz in LN-229 cells (FIGS. 3A and 4A) appeared to suggest that the relative location of the H.8 moiety, either in the N-terminal or in the C-terminal of Paz, did not affect its ability to promote internalization of the Paz moiety in glioblastoma cells.

### Example 4

### H.8 Moiety Promotes Paz Entry in Glioblastoma but not in Breast Cancer Cells

In order to determine if the H.8 epitope need to be a part of Paz, as in Laz, or could it function alone to promote Paz entry into glioblastoma cells, various H.8 fusion proteins, in addition to H.8 alone where used. Since small peptides such as the 39-amino acid synthetic H.8 moiety have low stability in solution, we constructed glutathione S-transferase (GST) fusions with the H.8 moiety, similar to Paz-H.8 or H.8-Paz, such that H.8 was incorporated in the N-terminal of GST (H.8-GST) or in the C-terminal of GST (GST-H.8). The construction of the GST fusion peptides is described under Example 1.

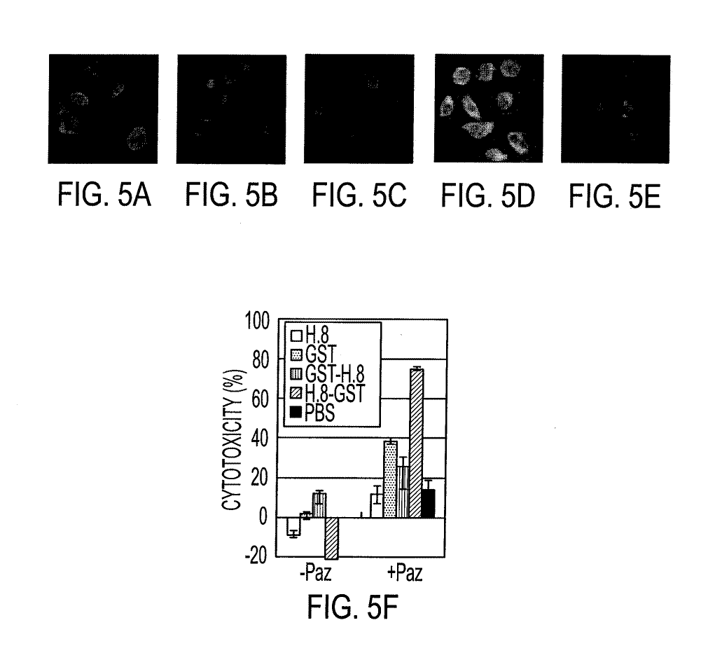

Alexa fluor® 568-conjugated Paz, fluorescing red, was incubated with unlabeled synthetic H.8 peptide, GST, GST-H.8 and H.8-GST fusion proteins separately, along with phosphate-buffered saline (PBS) as a control, and determined the internalization of 20 μM Paz mixture in LN-229 cells after incubation at 37° C. for 30 min. The synthetic H.8 peptide, when introduced separately along with Paz, did enhance Paz internalization (FIG. 5A) as compared to PBS (FIG. 5E), GST (FIG. 5B) or GST-H.8 (FIG. 5C). Quantification of the fluorescence showed that the H.8 peptide stimulated Paz entry by 2.1 fold. The presence of H.8-GST, however, significantly enhanced (more than 3 fold) the internalization of Paz (FIG. 5D). GST-H.8, on the other hand, showed only a mild stimulation (FIG. 5C). Paz itself entered only slowly (FIG. 5E) in glioblastoma cells, demonstrating that the entry in the brain tumor cells is mediated by H.8. H.8 alone did not enter the glioblastoma cells (FIG. 3A) but its ability to stimulate the internalization of Paz (FIG. 5A) reflects its ability to facilitate entry of proteins into brain tumor cells.

### Example 5

### Enhanced Internalization of Paz in Presence of H.8-GST in Glioblastoma Cells Lead to Higher Cytotoxicity in Such Cells

We incubated the synthetic H.8 peptide, GST, GST-H.8 and H.8-GST proteins (20 μM each) in absence or in presence of 20 μM Paz with LN-229 cells for 24 h and then measured the extent of cytotoxicity by measuring the viable glioblastoma cells by the MTT assay after 24 h. In absence of Paz, none of the H.8 peptide, GST or GST fusion proteins demonstrated any significant cytotoxicity (FIG. 5F, −Paz). In presence of 20 μM Paz, which itself demonstrated a low cytotoxicity in presence of the H.8 peptide or the PBS (FIG. 5F, +Paz), considerable enhancement of cytotoxicity was observed only in presence of the H.8-GST (FIG. 5F, +Paz), although GST itself or GST-H.8 did show some enhancement of cytotoxicity (FIG. 5F, +Paz). Taken together, these data suggest that the H.8 moiety, when present as part of or in presence of a protein such as Paz, facilitates the transport of Paz inside such cells, resulting in enhanced cytotoxicity.

### Example 6

### H.8 Mediate Crossing of the BBB to Allow Entry in Brain

The ability of the H.8 epitope to allow enhanced internalization of a fusion or individual protein in glioblastoma LN-229 cells (FIGS. 3A, 4A and 5D) raised the question of whether H.8 as part of the N-terminal of H.8-Paz or Laz promoted crossing of the BBB and allow transport of these proteins from peripheral circulation to brain venules.

Odyssey Assay. All proteins were labeled using IRDye® 800CW (LI-COR Biosciences, Lincoln, Nebr.) under conditions recommended by the manufacturers. Five hundred μg of Paz, H.8-Paz and Laz conjugated with IRdye® 800CW were injected intraperitoneally in nude mice. After 24 h, the mice were sacrificed, brains were taken out and brain images were detected with the LI-COR Odyssey® Infrared Imaging System (84 μm resolution, 1 mm offset). The mice brains were then cut horizontally and rostral mesencephalon region images were taken for detecting the presence of the labeled proteins.

Quantification of Fluorescence in Azurin Proteins. Quantification of fluorescence was measured by using Adobe® Photoshop® as follows: one cell was selected by Lasso Tool of Photoshop®, and the mean value was taken from red histogram of image menu. At least three different cells were measured for one sample and the standard deviation was calculated.

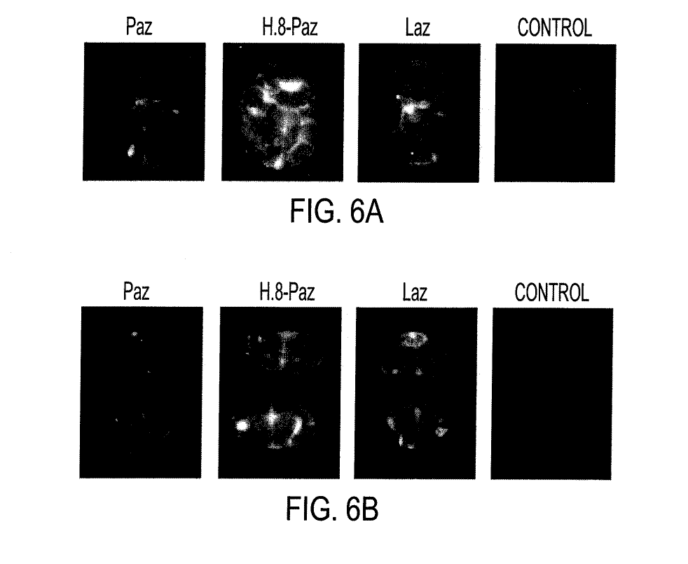

Five hundred μg of Paz, H.8-Paz and Laz proteins, labeled with the infrared dye IRdye® 800 CW (LI-COR Bioscience), was injected intraperitoneally into live nude mice. After 24 h, the mice were sacrificed, the brains were isolated and images were taken, using the LI-COR Odyssey® Infrared Imaging system. While Paz was found to enter the brain venules in small amounts, much more Laz and in particular H.8-Paz (more than 4 fold) was detected inside the brain under such conditions (FIG. 6), demonstrating a clear role of the H.8 epitope to allow entry of the fusion proteins into the brain.

### Example 7

### The H.8 Epitope, when Present in the N-Terminal, Allows Bacterial Surface Display of the Periplasmic Proteins

To investigate if the N-terminal localization of the H.8 epitope in Laz contributes to its surface display, H.8 fusion derivatives were constructed in the N- and C-terminals of GST (FIG. 5) and Paz (FIG. 2 and FIGS. 3A/B and 4A/B), as described in Example 1.

Localization of Surface-Exposed Proteins in E. coli. E. coli strain BL21 (DE3) harboring pET29a-gst, pET29a-H.8.gst or pET29a-gst-H.8 and E. coli strain JM109 harboring pUC19-paz, pUC19-paz-H.8, pUC18-H.8-paz or pUC18-laz were cultured at 37° C. with 0.4 mM Isopropyl β-D-thiogalactoside (IPTG). One ml each of these bacterial cultures was centrifuged and the resultant pellets were collected. After washing with PBS twice, one ml 1% FBS-PBS containing anti-GST antibody (1:2000) for GST derivatives or anti-azurin antibody (1:500) for azurin derivatives was added. Cell suspensions were incubated on ice for 1 h and then washed with PBS twice. FITC-conjugated anti-rabbit IgG for GST derivatives or FITC-conjugated anti-rabbit antibody for azurin derivatives was applied and incubated on ice for 30 min. To remove unbound antibody, cells were washed with PBS twice, and fixed with ethanol on ice. E. coli samples treated with DAPI were then observed under the confocal microscope.

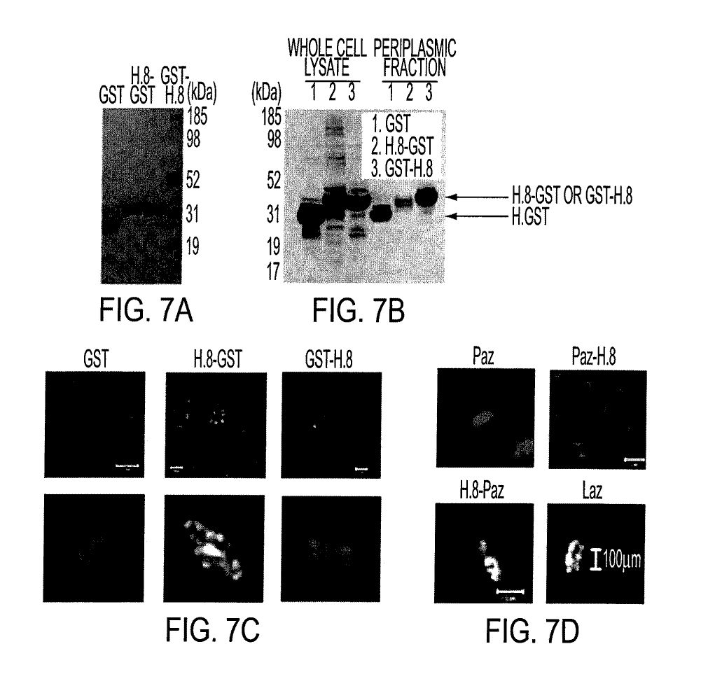

The H.8 fusion proteins were purified (FIG. 1E and FIG. 7A). The cellular localizations of GST, as well as the two H.8 fusions in the N- and C-terminals (H.8-GST and GST-H.8) are shown in FIG. 7B. All three proteins were hyper-expressed in E. coli and present in the whole cell lysates of E. coli, when detected by Western blotting using anti-GST antibodies (FIG. 7B). When the periplasmic fractions were isolated from E. coli and the presence of the three proteins checked, GST and GST-H.8 proteins were detected in significant amounts (FIG. 7B, lanes 1 and 3 under periplasmic fraction) but only small amounts of the H.8-GST (FIG. 7B, lane 2 under periplasmic fraction) could be detected in such periplasmic fractions.

To examine if the rest of the H.8-GST fusion protein might have been transported to the surface of E. coli cells, cells hyperexpressing the three proteins were grown and harvested, washed, treated with anti-GST antibody to bind any surface-exposed GST, washed again and treated with FITC-conjugated secondary antibodies. If the GST is surface-exposed, anti-GST antibodies would bind to them which then could be detected by FITC-conjugated secondary antibodies. Indeed, only E. coli cells harboring H.8-GST showed the FITC generated green fluorescence (FIG. 7C, H.8-GST), suggesting that the presence of the H.8 epitope in the N-terminal of GST promoted its transport to the cell surface. The presence of the H.8 moiety in the C-terminal of GST (GST-H.8), as well as GST itself, remained largely periplasmic and intracellular without any surface display (FIG. 7C, GST and GST-H.8).

Using the same technique as described above, it was determined that Paz and Paz-H.8 remained intracellular (FIG. 7D, Paz and Paz-H.8) while both H.8-Paz and Laz showed surface display, confirming that the presence of the H.8 in the N-terminal, perhaps requiring a free cysteine for lipidation, is important for transport of the fusion proteins through the outer membrane to reach the surface.

### Example 8

### Preparations of Azurins, Laz, and Pa-CARD Peptides for Leukemia Studies

Wildtype (wt) Azurin and mutant Azurin were purified as described in Yamada T et al., Proc Natl Acad Sci USA 99:14098-103 (2002) and Punj V et al., Oncogene 23:2367-78 (2004). Laz was purified using the same protocol as described in Hong C S et al., Cell Cycle 5:1633-41 (2006). In brief, Laz was expressed using a pUC18 vector in Escherichia coli. Cells were incubated for 24 h, spun down, and washed twice in PBS before being lysed for isolation of the periplasmic fraction. The periplasmic fraction is collected for Q sepharose exchange for protein purification. The fraction is concentrated and run on FPLC for isolation of the purified protein.

Cloning, expression and purification of Pa-CARD. The pET-SUMO expression vector which has SUMO fusion protein at its N82 terminal region was used for high level expression of soluble Arginine Deiminase and the caspase recruitment domain (CARD)-containing domain from P. aeruginosa ADI. To clone the CARD motif of the ADI gene into the pET-SUMO vector (Invitrogen), the gene was amplified from genomic DNA of a clinical isolate of P. aeruginosa with the following primer pair: F, fwd: 5′-ATGCACAATCTGCTGACCGAGACCATCCAG-3′ (SEQ ID NO: 28) and R, rev: 5′-TCAGGTCGAGGAGCCGTGGTCCTTGTC-3′ (SEQ ID NO: 29). The PCR product was directly ligated into a pET-SUMO TA cloning vector. The resulting expression vector was sequence confirmed and transformed into E. coli 90 strain BL21 (DE3).

Overnight cultures were grown in LB medium at 37° C. containing 50 μg/ml kanamycin. At OD₅₉₅nm, approximately 0.5, IPTG with a final concentration of 0.5 mM was added to the culture and incubated for 5 hrs at 37° C. Cells were collected by centrifugation at 5000 rpm for 15 min. Cell pellets were lysed by lysis buffer containing 50 mM Tris-Cl, 100 mM NaCl and 25% (w/v) sucrose, pH, 8.0. After addition of lysis buffer, 20 mg/ml lysozyme was added to cell suspension and incubated for 20 min at 4° C. Triton X-100 [final concentration 0.01% (v/v)] was added and incubated for 5 min. 100 μl DNase and RNase were added per 500 ml culture and incubated for 30 min at 37° C. The cell suspension was centrifuged at 15,000 rpm for 35 min at 4° C. The supernatant was loaded on pre-equilibrated 1 ml Ni-NTA column. After loading, the column was washed with buffer (50 mM Tris-Cl, 300 mM NaCl, 10% glycerol, 10 mM imidazole). SUMO-CARD was eluted with 25 ml step gradient of 50-500 mM imidazole in elution buffer (100 mM Tris-Cl, 500 mM NaCl, 20 mM imidazole, pH, 8.9). The purified SUMO-CARD fractions were pooled and exchanged with a buffer (20 mM Tris-Cl, 150 mM NaCl, pH, 8.0) for SUMO protease digestion. The SUMO-CARD protein was then concentrated up to 1.5-2 ml. Then DTT was added to a final concentration of 1 mM. After addition of SUMO protease, the digestion mixture was incubated at 30° C. for 2-3 h and the CARD polypeptide, called Pa-CARD, was purified by using nickel resin. The final protein concentration was measured with protein reagent (Pierce) using BSA as a standard. The ADI enzyme of P. aeruginosa, from which the CARD domain was derived, was purified from the SUMO-ADI fusion protein using an identical procedure (Pa-ADI).

Cell culture. All cell lines were cultured in RPMI 1640 medium supplemented with 10% fetal bovine serum (FBS), 2% penicillin/streptomycin, and 2% glutamine (Gibco/Life Technologies Inc.). All cells were grown in a humidified 37° C. incubator in 5% CO2.

Cytotoxicity assay. The MTT [3-(4,5 dimethylthiazol-2-yl-2,5 tetrazolium bromide)] assay was used for the measurement of the cytotoxicity of wt and mutant Azurin as described Yamada T et al., Proc Natl Acad Sci USA 99:14098-103 (2002) and Punj V et al., Oncogene 23:2367-78 2004). In brief, the water soluble tetrazolium salt, [3-(4,5 dimethylthiazol-2-yl-2,5 tetrazolium bromide)] is metabolized to the water insoluble formazan by intact mitochondrial dehydrogenases. The formazan is then solubilized by adding 2-propanol+40 mM HCl for a 1-h incubation. 3×10⁴ cells were treated with varying concentrations of Azurin, Laz or Pa-CARD, as well as 5-aza-2-deoxycytidine (DAC) as a positive control. The viability of the cells was estimated on the basis of formazan formed, which was detected spectrophotometrically by optical density at 570 nm.

Cell cycle analysis. HL60 and K562 cells (3×10⁵ cells seeded per well in 24-well plates) were treated with wild type Azurin, Pa-CARD, or Laz (5 and 10 μM) for 48 h. The cells were washed twice with PBS and fixed with 70% ethanol for 24 h at −20° C. Fixed cells were washed twice with PBS and stained with 50 μg/ml of propidium iodide in PBS containing 20 μg/ml RNase A for 30 min in the dark and analyzed by flow cytometry (Becton Dickinson). The percentage of cells in different phases of the cell cycle was determined by MODFIT LT software.

Western blotting. For immunoblotting, cytoplasmic and nuclear fractions were isolated using the NE-PER extraction reagent (Pierce) according to the manufacturer's protocol, with complete protease and phosphatase inhibitors (Sigma). Protein concentrations of the cell lysates were measured using the Bradford Bio-Rad assay. Cell lysate proteins were separated via SDS-PAGE and transferred to a PVDF membrane for immunoblotting. Membranes were blocked in Tris-buffered saline (0.15 M NaCl, 0.05 M Tris-HCl [pH 8.0], 0.05% Tween 20) containing 5% nonfat dry milk (Difco) and incubated overnight at 4° C. with primary antibody (at recommended dilutions in TBST, 5% nonfat dry milk) with gentle agitation. After three washes for 5 min each with TBST, the membranes were probed with horseradish peroxidase-labeled goat anti-rabbit or rabbit anti-mouse antibody from Zymed Laboratories (San Francisco, Calif.) (1:3000 in TBST, 5% nonfat dry milk) for 1 h at room temperature. After additional washing steps, membranes were incubated with a chemiluminescent substrate for 1 min at room temperature. To remove bound antibody between each antibody incubation, membranes were incubated in Restore Western blot stripping buffer (Pierce) according to the manufacturer's protocol and reprobed. Anti-B-actin was from Sigma. AKT, Wee1, and anti-phospho-AKT-S 473 antibodies were from Cell Signaling Technology (Beverly, Mass.). The secondary antibodies, horseradish peroxidase-labeled goat anti-rabbit and rabbit anti-mouse was from Zymed Laboratories (San Francisco, Calif.).

Confocal microscopy. Azurin and Laz proteins were conjugated with the fluorescent chemical AlexaFluor 568 (Molecular Probes) and incubated with HL60, K562, or normal peripheral blood mononuclear cells for 1 h. Entry of fluorescent chemically labeled peptide or protein into the cells was observed by confocal microscopy (model LC510, Carl Zeiss), as described in Yamada T et al., Cell Microbiol. 7:1418-31 (2005).

### Example 9

### The effects of Azurin and Laz on Leukemia Cells

Bacterial proteins Laz and Azurin were examined for their ability to induce cell cytotoxicity and thereby reduce viability of two leukemia cell lines, HL60, an AML cell line and K562, a CML cell line. Viability of cell lines HL60 and K562 were measured by their ability to metabolically reduce MTT to a purple formazan product after 24 h, 48 h and 72 h of treatment with Laz or Azurin at 10 μM (FIGS. 1A and B). 10 μM of protein turned out to be highly cytotoxic, reducing cell viability by more than 90%. Laz is a Neisserial protein with an Azurin component, which has substantial sequence identity with P. aeruginosa Azurin, but with an additional 39 amino acid peptide called an H8 epitope.

To determine if this H8 epitope modulates anticancer activity of Azurin, the H.8 epitope was cloned in the N- and C-terminus of P. aeruginosa Azurin, giving rise to H8-Azurin (H8-Azu) and Azu-H8. Hong C S et al., Cell Cycle 5:1633-41 (2006). These two Laz-like proteins were also tested against the leukemia cell lines. To determine a lower effective dose, K562 cells were treated with varying concentrations of protein, with final concentrations at 3.75 nM and upwards, while HL60 cells were treated with 1-10 μM final concentrations. All the bacterial proteins exhibited a dose dependent cytotoxic effect on the two leukemia cell lines. Significant cell viability reduction was observed with even nanomolar (nM) protein concentrations, with peak reduction in viability with 5 μM of protein (FIGS. 8C and D). This cytotoxic effect was apparent after 24 h of treatment with all the proteins exerting cytotoxic effects in both HL60 and K562 cells (FIGS. 8A and B).

The cytotoxic effects of Laz and Azurin were also compared to a known leukemia drug, 5-aza-2′-deoxycytidine (DAC). 5-Aza-2′-deoxycytidine is a DNA methylation inhibitor that acts by reactivating tumor suppressor genes that are silenced by DNA hypermethylation. Reactivation of tumor suppressor genes leads to cell cycle arrest and apoptosis. The viability reduction effects of each bacterial protein were significantly better than that of DAC (FIGS. 8C and D). Azurin or Laz are cytotoxic to cancer cells without being cytotoxic to normal cells. This cytotoxicity has also been shown to be due to the peptide itself, and not due to cellular contaminants such as endotoxins.

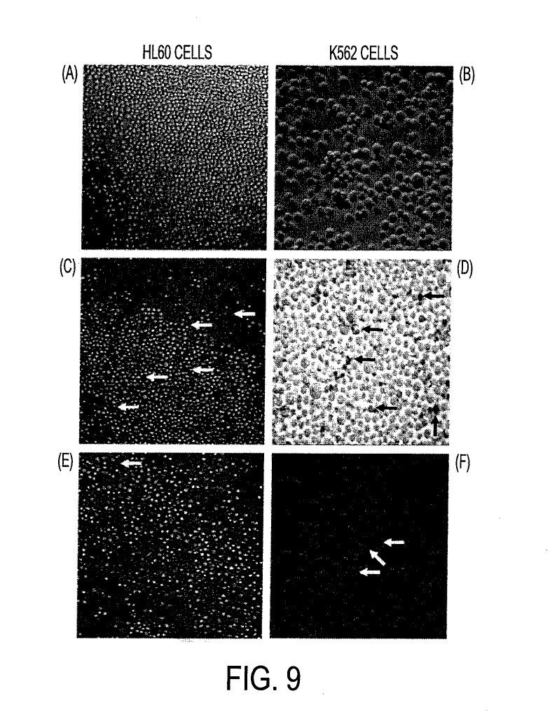

In an effort to distinguish the possible mechanisms of cell death induced by Laz and Azurin, apparent gross morphological changes were searched for via fluorescent microscopy. Changes were looked for that would help distinguish between cell necrosis, apoptosis, and cellular differentiation, processes that would ultimately lead to cell death. Changes such as granulation or cellular enlargement are indicators of possible differentiation or necrosis and ultimate cell death in both cases for acute myeloid and chronic myeloid leukemia cells. Fluorescent microscopic results show that Laz and Azurin are able to induce cellular differentiation in HL60 and K562 cells after 48 h of incubation 210 at 10 μM concentration (FIG. 9).

### Example 10

### The entry of Laz and Azurin into HL60 and K562 Cells

The ability of Azurin and Laz to enter HL60 and K562 cells was examined using confocal microscopy and AlexaFluor 568-conjugated red fluorescent Azurin or Laz. These two proteins did not appear to enter normal PBMCs (FIGS. 10A and B, panel 1), but there was significant entry of Laz and Azurin into both K562 (FIGS. 10C and D, panel 1) and HL60 cells (FIGS. 10E and F, panel 1). Azurin showed lesser entry (FIGS. 10D and F, panel 1). The level of cytotoxicity of Azurin (FIGS. 8C and D) at 1.0-2.5 μM might be linked to its level of entry in HL60 or K562 cells.

The entry of H8-conjugated Azurin, Azu-H8 and H8-Azu was examined, which demonstrated similar or somewhat higher cytotoxicity than Azurin, particularly at lower concentrations such as 1.0-2.5 μM, but comparable to Laz (FIGS. 8C and D). Both Azu-H8 and H8-Azu showed higher level of entry than Azurin, comparable to that of Laz in K562 cells (FIG. 10, panel 2), demonstrating a role of the H8-epitope in allowing entry of Azurin or Laz in leukemia cells.

### Example 11

### Laz Causes Cell Cycle Arrest in K562 Cells by Increasing Wee1 Protein Levels and Decreasing Phosphorylated AKT

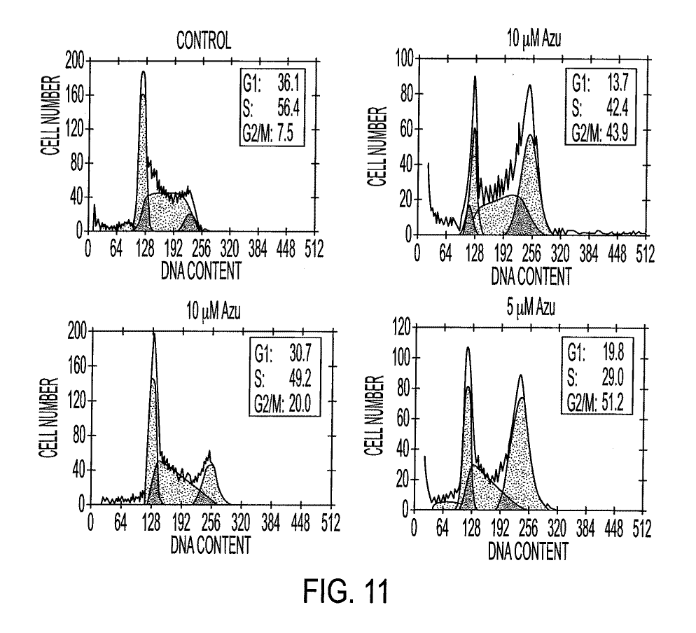

The effect of Laz, Azurin and or 5-aza-2-deoxycytidine (DAC) on cell cycle progression was evaluated using flow cytometric analysis in K562 cells, which seemed to be highly susceptible. The effect of Laz, Azurin and DAC on K562 cell cycle arrest was observed (FIG. 11). Laz, Azurin and DAC are able to arrest K562 cells at G2/M. Cell cycle arrest at G2/M phase often leads to induction of apoptosis and cell death, as has been observed for Azurin and Laz. It is possible that proteins such as Laz and Azurin can influence the levels and or activity of G2 cell cycle regulators in leukemia cells.

The effect of Laz on regulators of the cell cycle that mediate G2 arrest including Wee1 and phosphorylated AKT was analyzed. Wee1 protein is known to be a mediator of cell cycle arrest at G2/M phase. Many viral proteins have been shown to be able to arrest cell cycle at the G2 phase by interacting with and influencing Wee1 activity in vivo. Treatment of K562 cells with 10 μM Laz led to an increase in Wee1 protein levels in both the cytoplasm and the nucleus (FIG. 12), where Wee1 is known to exert its effects. In contrast to K562 cells, the HL60 cells showed a significantly reduced level of Wee1 protein. Inhibiting phosphorylation of AKT at serine 473 and hence AKT activity is also associated with G2/M cell cycle arrest. Laz treatment also led to a significant decrease in AKT activity in HL60 cells as evidenced by a decrease in the levels of phosphorylated AKT serine 473 (FIG. 12). Interestingly, the decrease in AKT-P-Ser 473 levels were more pronounced in HL60 cells than in K562 cells, while the changes in nuclear Wee1 protein levels by Laz were more pronounced in K562 cells.

### Example 12

### Pa-CARD, an Anticancer Bacterial Protein, has Activity Against Leukemia Cells

M. arginini ADI has been reported to inhibit the proliferation of cultured human lymphatic leukemia cells by causing growth arrest in the G1/S phase, leading to apoptosis. Gong H et al., Leukemia 14:826-9 (2000). ADI is believed to exert its action by depleting arginine, converting arginine to citrulline and ammonia. Arginine is a non-essential amino acid since mammalian cells can resynthesize it back from citrulline. However, certain cancers such as hepatocellular carcinoma, melanoma or renal cell carcinomas do not express in vivo argininosuccinate synthetase, an enzyme important for synthesizing arginine from citrulline, making such cancer cells uniquely sensitive to arginine deprivation due to ADI action.

An interesting feature of M. arginini ADI is the discernible structural similarity of its N-terminal part to mammalian CARD proteins. The eukaryotic, primarily mammalian, CARD proteins carry a domain called caspase recruitment domain which is a protein-protein interaction motif. The presence of CARD domains in CARD-carrying proteins allows formation of complexes through CARD-CARD interactions, leading to activation/inhibition of cell signaling mediated by NF-kβ or processes leading to cell death. CARD-carrying proteins such as caspases are not only involved in the induction of apoptosis but also in proinflammatory cytokine production. Thus, a caspase such as caspase 1 is involved in the proteolytic processing of a cytokine such as IL-1B to allow its release from the cell in the activated form. Since IL-1B promotes angiogenesis and is hyperproduced in many tumors, CARD-carrying proteins such as caspase 1 are considered major targets for inhibitor development as anticancer agents.

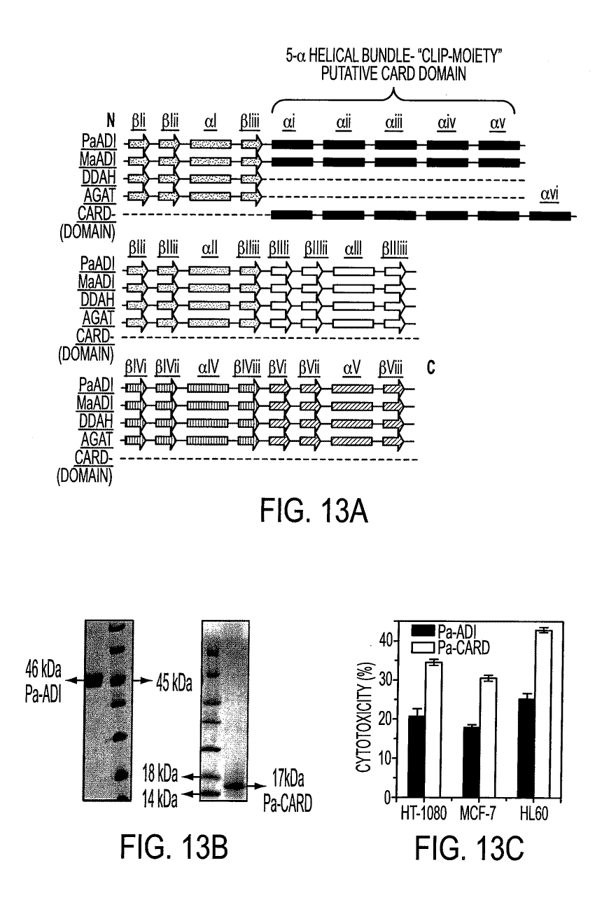

The gene sequences and crystal structures of ADI from M. arginini and P. aeruginosa, called Ma-ADI and Pa-ADI, respectively, are known, and demonstrate about 27% sequence identity with the presence of a common CARD motif in the N-terminal end (FIG. 13A). ADI is a member of a guanidine-modifying enzyme (GME) superfamily with the ability to catalyze nucleophilic substitution reactions at the guanidinium carbon atom similar to other family members such as dimethylarginine dimethylaminohydrolase (DDAH) and arginine:glycine amidinotransferase (AGAT). Alignment of the structural features of the GME superfamily members, however, demonstrates the presence of the CARD motif only in ADI but not in other members (FIG. 13A), demonstrating the recruitment of the CARD domain by ADI with implications of its potential role as an anticancer agent associated with it

The region from P. aeruginosa ADI having the structural features of CARD (amino acids 75-225) was cloned in frame with the SUMO moiety from the plasmid pET-SUMO, giving rise to the SUMO-CARD fusion protein. This fusion protein was purified using Ni-NTA column and the CARD polypeptide was isolated as a homogeneous 17 kDa protein (FIG. 13B). The activity of this protein, called Pa-CARD because of its origin from P. aeruginosa ADI N-terminal CARD domain, as well as the ADI from P. aeruginosa (Pa-ADI, 46 kDa, FIG. 13B),was then determined using solid tumor cell lines, fibrosarcoma HT-1080, breast cancer MCF-7 and the leukemia cell line HL-60.

Pa-CARD had significant cytotoxic activity against HT-1080, MCF-7 and leukemia HL60 (FIG. 13C) as well as other cancers, but less so for normal fibroblasts and normal breast cells such as MCF-10A (data not shown). Since M. arginini ADI has been reported to inhibit human leukemia cell proliferation by inducing cell cycle arrest, the ability of Pa-CARD to induce cell cycle arrest in the cancer cells was also measured. Pa-CARD demonstrated significant arrest of the cell cycle (data not shown), as previously reported for Laz and Azurin (FIG. 11). Similar to Laz, Pa-CARD enhanced Wee1 protein level in the nuclear fraction of K562 cells but did not demonstrate such an activity in the HL60 cells (FIG. 12). However, in nuclear fractions of HL60 cells, and in the cytoplasmic fractions of K562 and HL60 cells, the AKT-P-S473 levels were significantly reduced by Pa-CARD (FIG. 12), suggesting that the primary mode of cell cycle arrest in HL60 cells by Pa-CARD might be mediated through reduction of the 473 serine phosphorylation of AKT, while in K562 cells, such an effect is primarily mediated through enhancement of nuclear Wee1 protein levels, although the cytoplasmic 473 serine phosphorylation of AKT is also affected to some extent. Laz mimics Pa-CARD in mediating such effects (nuclear Wee1 enhancement in K562 and cytoplasmic AKT-S473 depletion in HL60 cells), suggesting a common mode of action.

### Example 13

### Materials and Methods for Ovarian Cancer Study

Chemicals, Reagents and Cell Cultures: MTT(3-(4,5-Dimethylthiazol-2-yl)-2,5-diphenyltetrazoliumbromide) and propidium iodide were purchased from Sigma. Prestained gels were obtained from Biorad. pET SUMO kit and SUMO Protease were purchased from Invitrogen. The human cancer and normal cell lines SK-OV3, HOSE6-3, MCF-7, and HT1080 cells are all from the stock culture collection of the Department of Surgical Oncology, University of Illinois at Chicago (UIC), Chicago. The cells were cultured in MEM with Eagle's salt containing 2 mM L-glutamine, 0.1 mM MEM essential amino acids and supplemented with 10% heat-inactivated fetal bovine serum, 100 units/ml penicillin and 100 μg/ml streptomycin. All cells were grown at 37° C. in 5% CO2.

Cloning, Expression, and Purification of Proteins: performed as described in Example 8 herein.

MTT-Cytotoxicity Assay: The 3-(4,5-dimethylthiazol-2-yl-2,5-diphenyl)tetrazolium bromide (MTT) assay was performed as described in Example 8 to determine the cytotoxicity toward cancer cells.

Caspase-3/7 Activity Assay: Tests were performed in 96-well plates using caspase-Glo 3/7 (Promega) reagent as described in Yamada, T. et al. Proc. Natl. Acad. Sci. U.S.A, 2002, 99, 14098-14103.

Cell Cycle Arrest by Flow Cytometry: SKOV3 cells were seeded in 6 well plate at 10⁶ cells per well in a volume of 2 ml per well. Cell pellets (>1×10⁶ cells), were harvested and were fixed in 1 mL of 70% ethanol at 4° C. for 60 min, washed in 1 mL of PBS and resuspended in 400 L of PBS containing 0.5 mg of RNAse A (Sigma). After gentle mixing, a 100-μL aliquot of propidium iodide (1 g/L PBS) (Sigma) was added. The cells were incubated in the dark at room temperature for 15 min and then held at 4° C. in the dark for flow cytometric analysis. For each sample, at least 1×10⁴ cells were analyzed for DNA content using a Becton-Dickinson FACS Calibur Flow Cytometer. The distribution of cells in sub-G1, G1, S and G2-M was determined using Multi Cycle AV software.

Microarray of TLR Pathway and NF-kB Signaling Genes: SKOV3 cells (100,000 cells per well in a volume of 2 ml per well) were seeded on 6 well plate. Following treatment with proteins, cells were harvested with lysis buffer. The samples were sent to Superarray Biosciences for microarray of toll like receptor genes and NF-kB signaling genes.

TUNEL Assay: This technique identifies nuclei containing fragmented DNA by enzymatically incorporating fluoresceine-dUTP at the 3′ end of DNA fragments using terminal deoxynucleotidyl transferase as described in Yamada, T. et al. Proc. Natl. Acad. Sci. U.S.A, 2002, 99, 14098-14103.

### Example 14

### ADI has a Putative CARD-Like Domain

Secondary structure comparisons demonstrated 27% sequence identity between Pa-ADI and Ma-ADI. Using the MolMol program and VAST algorithm, structural alignments clearly demonstrate the presence of both the putative CARD-like domain and the catalytic domain in Pa-ADI and Ma-ADI (FIGS. 14A and B). Both Pa-ADI and Ma-ADI has the characteristic five ββ\acute{α}β-subunits present in all the proteins of the guanidine-modifying enzyme superfamily of which Dimethylarginine dimethylaminohydrolase (DDAH) and Arginine: glycine amidinotransferase (AGAT) proteins are members (FIG. 14B). However, unlike DDAH and AGAT, both Pa-ADI and Ma-ADI have a unique 85 residue 5-alpha helical bundle domain inserted precisely between the first and the second ββ\acute{α}β-subunits giving the typical “Clip-on-fan” moiety (FIGS. 14B and 14C). It is this region which exhibits structural similarity to the mammalian CARD-containing proteins. Since the CARD-like domain is present only in the Pa-ADI and Ma-ADI but not in the other members (FIG. 14B), the recruitment of the CARD domain by ADI but not by other members of the family likely gives ADI its unique anticancer property.

### Example 15

### Pa-CARD has Anticancer Activity

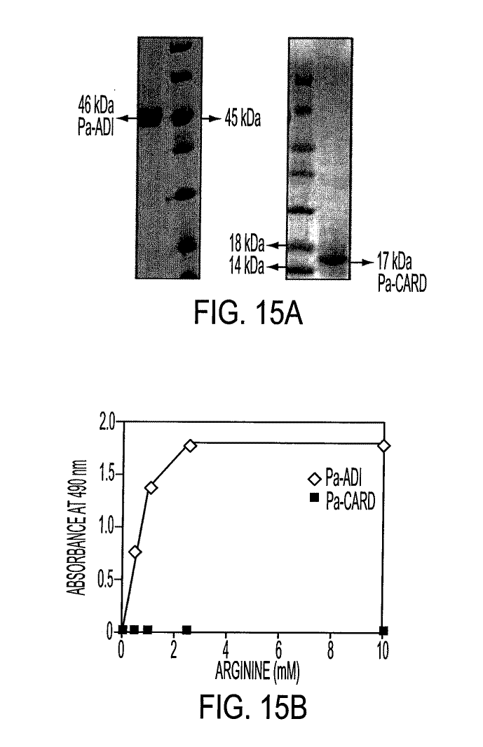

The CARD containing mammalian proteins have diverse functions, such as helicase, kinase and caspase activity. The CARD domain by itself is not known to have any other activity besides mediating formation of larger protein complexes via CARD-CARD interactions. Assuming that bacteria compete with cancer cells for the same nutrition inside the body as a source of its survival and proliferation, it is possible that bacteria acquired the CARD domain from a mammalian source, presumably to interfere in cancer growth. To test this hypothesis, the putative CARD domain with the hydrophobic pocket from Pa-ADI (amino acids 75-225) was cloned and purified, and its function as an anticancer agent as well as for any potential ADI-like activity was examined. The gene sequences of Pa-ADI encompassing amino acids 75-225 as well as the full-length ADI protein were cloned in frame with SUMO moiety from plasmid pET-SUMO (Invitrogen), giving rise to SUMOCARD fusion and SUMO-ADI proteins, as described in Example 8. These recombinant proteins were purified in a single step using a Ni-NTA column, followed by cleavage with SUMO protease. The homogeneity and SDS-PAGE migration patterns of these two bacterial proteins are shown in FIG. 15A. As expected, the purified Pa-ADI migrated as a single band of Mw 46 kDa, while the Pa-CARD migrated as a 17 kDa protein.

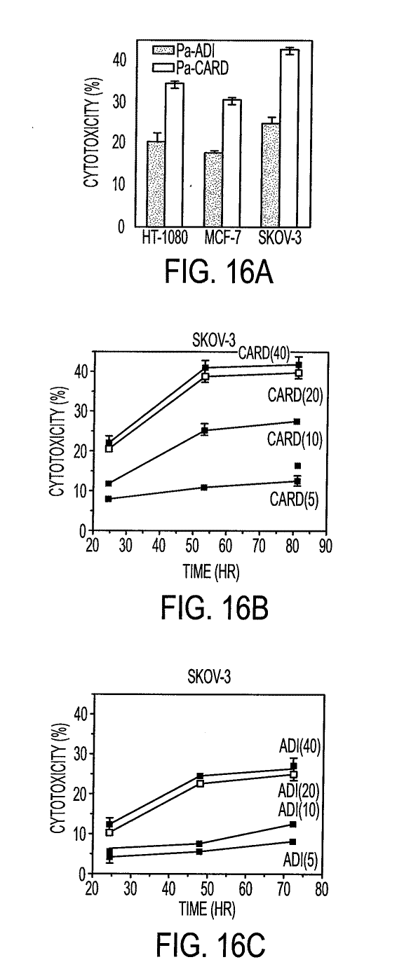

The arginine deiminase activity of these two proteins was determined next. As expected, Pa-ADI showed strong enzymatic activity while Pa-CARD had no such activity (FIG. 15B), since the critical amino acids Cys-406 and His-278 of the catalytic triad of Pa-ADI are missing in Pa-CARD. The M. arginini ADI (Ma-ADI) is known to have anticancer activity, although the presence of such activity has not been assessed in Pa-ADI or Pa-CARD. The growth inhibitory activities of Pa-ADI and Pa-CARD were checked against a range of human cancer cells such as fibrosarcoma HT-1080, breast cancer MCF-7 and ovarian cancer SKOV-3 (FIG. 16A). Both Pa-ADI and Pa-CARD demonstrated significant inhibitory activities against all the cancer cell types. Most interestingly, Pa-CARD had higher growth inhibitory activities than Pa-ADI against these cancer cells (FIG. 16A). Since Pa-ADI and Pa-CARD demonstrated somewhat higher activity against the ovarian cancer SKOV-3 cells, the cytotoxicity of various concentrations of Pa-CARD (FIG. 16B) and Pa-ADI (FIG. 16C) as was determined a function of time of incubation with these cells. While the cytotoxic activities were apparent during 24 hours of incubation, the activities were elevated significantly at higher concentrations (10 to 20 μM) during 48 hours of incubation. Once again Pa-CARD showed higher cytotoxicity compared to Pa-ADI in a concentration and time dependent manner.

### Example 16

### Pa-CARD has Very Little Inhibitory Effect on Normal Cells

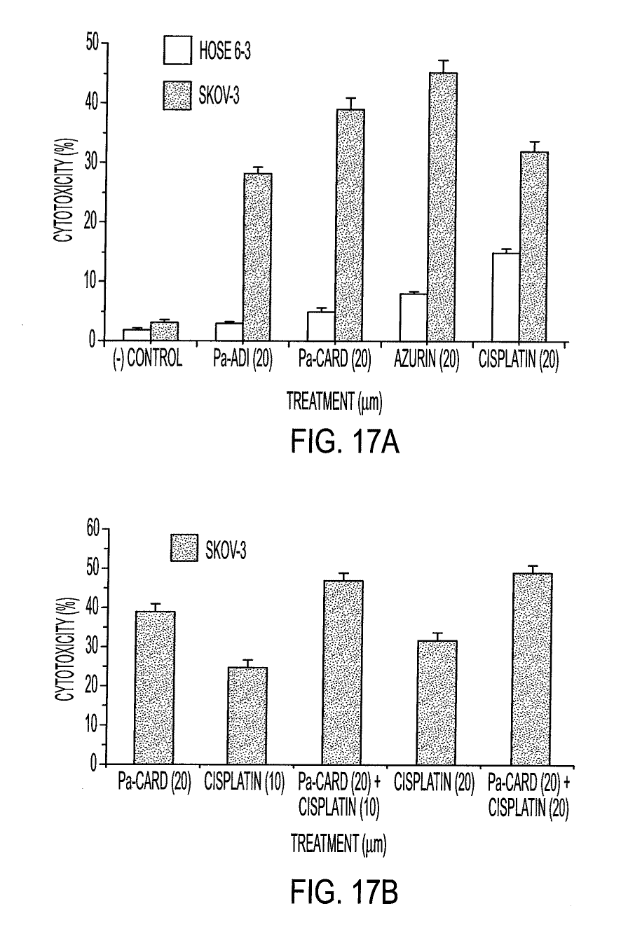

The cytotoxic effects of Pa-ADI and Pa-CARD at 20 μM concentrations on both ovarian cancer SKOV-3 and the normal ovarian HOSE6-3 cells were examined. Because azurin has anticancer activity against a range of cancers, but cannot not enter normal cells and demonstrates little cytotoxicity against such cells, 20 μM of azurin was added as a control in these studies. A known anticancer agent ‘cisplatin’ (cis-diamminedichloroplatinum (II); CDDP) was used as a positive control. The cytotoxic activities of these agents against HOSE6-3 normal ovarian and SKOV-3 ovarian cancer cells are shown in FIG. 17A. Both Pa-ADI and Pa-CARD have significant cytotoxic activity against SKOV-3 but very little cytotoxicity towards the normal ovarian cells. Azurin also demonstrated a similar cell death profile. Both Pa-CARD and azurin demonstrated lower cytotoxicity to the normal HOSE6-3 cells than cisplatin, a commonly used drug. Moreover, SKOV-3 cells treated with both Pa-CARD and cisplatin show somewhat higher cytotoxic effect as compared to when treated individually (FIG. 17B), showing some additive effects.

### Example 17

### Pa-CARD Induces Apoptosis in Cancer Cells through Caspase Activation

To determine the nature of cell death induced by Pa-CARD, and since other bacterial proteins such as azurin and Laz are known to induce apoptosis in cancer cells through caspase activation, the extent of induction of apoptosis by Pa-CARD, Pa-ADI and azurin in both ovarian cancer SKOV-3 and normal ovarian HOSE6-3 cells was determined by TUNEL assay using the in situ Cell Death Detection Fluorescein Kit (Roche). When the SKOV-3 or the HOSE6-3 cells were treated with DNase I, essentially all cells fluoresced green (Positive control, FIG. 18). When both the SKOV-3 cells and HOSE6-3 cells were treated with 10 μM of Pa-CARD, Pa-ADI and azurin, enhanced apoptosis was evident selectively in the SKOV-3 cells. Also highest percentage of apoptosis was observed when SKOV-3 cells were treated with Pa-CARD as compared to Pa-ADI and azurin. Since apoptotic cells tend to compromise their adherent property, fewer cells are seen in slides with higher percentage of apoptotic cells (FIG. 18). HOSE6-3 cells showed only a few cells fluorescing green (FIG. 18) under all conditions, confirming that Pa-CARD induces apoptotic cell death primarily in the cancer cells and not in the normal cells. Treatment with bovine serum albumin (BSA) or no protein treatment (control) showed very few green fluorescing cells and many blue (non apoptotic cells) in the field (FIG. 18, bottom row), demonstrating that the cancer or normal cells do not spontaneously undergo much apoptosis, unless treated by Pa-CARD, Pa-ADI or azurin.

To determine if Pa-CARD induces cell death in ovarian cancer cells but not in normal ovarian cells through caspase 3 activation, the levels of caspase 3/caspase 7 in SKOV-3 and HOSE6-3 cells were measured in absence or in presence of Pa-ADI, Pa-CARD, Cisplatin and azurin. The caspase enzyme activity was determined by the Caspase-Glo™ 3/7 assay kit (Promega). Pa-ADI and Pa-CARD were used in two different concentrations, 10 and 20 μM. Both Pa-ADI and Pa-CARD induced caspase 3/7 activation in a dose dependent manner, but only in the ovarian cancer SKOV-3 cells. Very little activation was seen in normal ovarian cells HOSE6-3 (FIG. 19).

### Example 18

### Pa-CARD Inhibits Cell Cycle at the G2/M Phase

Mycoplasma arginini ADI (Ma-ADI) has previously been shown to inhibit the growth of hepatocellular carcinomas and to induce cell cycle arrest and apoptosis in leukemia cells. The induction of apoptosis through caspase activation is often known to be mediated by cell cycle arrest at the G2/M phase including in hepatoma, leukemia and other cancers.

The levels of G1, S and G2 phases in cancer cells treated with or without Pa-ADI and Pa-CARD were examined. When SKOV-3 cells were treated with Pa-ADI for 24 hours at 10 μM concentration, cells at the G1 phase dropped from 86.0% to 69.1% while those at the S and G2 phases went up from 7.9 to 19.7 and 6.1 to 11.2% respectively. However, treatment with Pa-CARD under similar conditions reduced cells at G1 phase levels from 86.0 to 19.1% while the S and G2 phases in cells rose from 7.9 to 43.5% and from 6.1 to 37.4% respectively (data not shown), clearly showing that the Pa-CARD profoundly inhibited cell cycle at the G2/M phase.

### Example 19

### Pa-CARD Modulates Expression of NF-kB Signaling Pathway Genes

The effect of Pa-CARD in SKOV-3 cells was examined by measuring the expression of several genes involved in NF-kB signaling pathway. The goal was to identify candidate genes that may play a vital role in determining the fate of the SKOV-3 cells following treatment with Pa-CARD protein. For this study, SKOV-3 cells were incubated for 48 hours with 10 μM each of Pa-CARD and azurin and a control without any treatment. The total RNA was isolated to perform quantitative real time PCR microarray. The fold regulation was calculated by normalizing with the expression of the housekeeping genes. Only changes that were more than two fold control levels are reported.

A striking observation in the expression profile of the genes was an overall upregulation of most of the genes of NF-kB pathway including Toll-Like Receptors (TLRs) when SKOV-3 cells were treated with Pa-CARD as against azurin (Table 3). This is due to Pa-CARD-mammalian CARD interaction in SKOV-3 cells. Mammalian CARD-carrying proteins are known to modulate NF-kB signaling pathways and any interaction with Pa-CARD is likely to alter such gene expression profiles as observed. Azurin, on the other hand, is known to inhibit cancer cell growth through protein-protein interaction with p53 and receptor tyrosine kinases but not through modulation of NF-kB pathway. Thus these two bacterial anticancer proteins have two different modes of action.

The most significant up-regulation of the genes by Pa-CARD in SKOV-3 cells involves a 55 fold increase in the expression of the gene encoding colony stimulating factor CSF2, also known as granulocyte-macrophage colony-stimulating factor (GM-CSF). The expression of IL-12 gene is also stimulated by about 3 fold under such conditions (Table 3). Western blotting data (not shown) confirmed the hyperproduction (about 5 fold) of GM-CSF under such conditions. The levels were lower than a 55 fold increase, presumably because GM-CSF is a secretable protein that comes out of the cell. Other cytokines whose expressions are up-regulated during Pa-CARD treatment are IL-2, IL-8, IL-10, IL-1 alpha and IL-1 beta (Table 3).

TABLE 3
Measuring mRNA Levels of Toll-Like Receptor (TLR) and NF-kB
Pathway Genes Using Real-Time PCR Microarray: SKOV-3 cells
were incubated for 48 hours with 10 μM each of CARD and
azurin. As a control, one sample of SKOV-3 cells was left untreated.
After incubation, cells were harvested and total RNA was extracted.
The samples were analyzed using real-time PCR microarray analysis
(Superarray Bioscience). Results were normalized to housekeeping
genes. Values in bold indicate fold up regulation and numbers
marked with asterisk (*) indicate down regulation relative to untreated
control samples. Decreased expression is indicated with a minus sign.

Fold Up-or Down-Regulation

| | | |
|---|---|---|
| Gene symbol | CARD/Control | Azurin/Control |

NF-KB Pathway-Fold Regulation

| | | |
|---|---|---|
| CSF2 | 55.72 | 9.19 |
| CSF3 | 2.46 | 1.07 |
| IFNβ1 | 2.30 | −2.30* |
| IFNγ | 2.46 | 1.07 |
| IL10 | 2.46 | 1.07 |
| IL12α | 2.83 | 2.00 |
| IL1α | 10.56 | −1.62 |
| IL1β | 25.99 | 2.14 |
| IL2 | 2.46 | 1.07 |
| IL8 | 3.25 | 1.87 |
| NF-κB2 | 5.28 | 1.07 |
| NF-κBIL1 | 5.66 | −1.15 |
| CCL2 | −17.15* | −2.00 |
| MAP3K1 | −2.14* | −1.00 |
| MAP4K4 | 6.06 | 1.74 |
| RELA | 2.30 | −1.32 |
| TNFRSF1A | 2.46 | 1.15 |

Toll-Like Receptors, Adaptors & TLR Interactiong
proteins-Fold Regulation

| | | |
|---|---|---|
| TLR1 | 2.14 | −1.07 |
| TLR10 | 2.46 | 1.07 |
| TLR3 | 4.92* | −1.07 |
| TLR6 | 6.06 | 1.74 |
| TLR7 | 2.46 | 1.07 |
| TLR8 | 2.46 | 1.07 |
| TLR9 | 3.25 | 1.07 |
| LY86 | −2.14* | −4.92* |
| TICAM2 | 2.64 | −1.15 |
| BTK | 2.46 | 1.07 |
| CD14 | 4.00 | 1.52 |
| HSPA1A | 2.64 | 1.32 |

TLR Effectors-Fold Regulation

| | | |
|---|---|---|
| EIF2AK2 | 1.23 | 1.07 |
| IRAK1 | 2.64 | −1.32 |
| IRAK2 | 2.83 | 1.23 |
| PPARA | 3.73 | −2.00 |
| UBE2V1 | −3.48* | −1.00 |
| MAP3K7IP1 | 2.14 | −1.15 |

What is claimed is:

1. An isolated peptide that is capable of killing cancer cells, which comprises a caspase recruitment (CARD)-like domain.

2. The isolated peptide of claim 1, which is derived from a bacteria.

3. The isolated peptide of claim 2, wherein the bacteria is Pseudomonas aeruginosa.

4. The isolated peptide of claim 1, which is Pa-CARD.

5. The isolated peptide of claim 1, which comprises SEQ ID NO: 27.

6. The isolated peptide of claim 5, which consists of SEQ ID NO: 27.

7. The isolated peptide of claim 1, wherein the cancer is selected from the group consisting of leukemia, ovarian cancer, fibrosarcoma, and breast cancer.

8. The isolated peptide of claim 1, which is chemically modified to extend or optimize its half-life in the bloodstream.

9. A method, comprising killing cancer cells by contacting the cells with one or more proteins selected from the group consisting of the isolated peptide of claim 1, Laz, H8-Azu, and Azu-H8.

10. The method of claim 9, wherein the cancer cells are selected from the group consisting of leukemia cells, fibrosarcoma cells, ovarian cancer cells, and breast cancer cells.

11. The method of claim 9, also comprising contacting the cells with one or more cytotoxic agents that are capable of killing cancer cells.

12. The method of claim 11, wherein the one or more cytotoxic agents are selected from the group consisting of cisplatin, Gleevec®, Retinoic acid, 5′-aza-2′-deoxycytidine, and arsenic trioxide.

13. The method of claim 12, wherein the one or more cytotoxic agents is cisplatin.

14. The method of claim 11, wherein the cancer cells are contacted with the one or more cytotoxic agents at about the same time as the one or more proteins.

15. A method, comprising administering to a mammalian patient suffering from cancer one or more proteins selected from the group consisting of the isolated peptide of claim 1, Laz, H8-Azu, and Azu-H8.

16. The method of claim 15, wherein the cancer is selected from the group consisting of leukemia, fibrosarcoma, ovarian cancer, and breast cancer.

17. The method of claim 15, also comprising administering to the patient one or more cytotoxic agents that are capable of killing cancer cells.

18. The method of claim 17, wherein the one or more cytotoxic agents are selected from the group consisting of cisplatin, Gleevec®, Retinoic acid, 5′-aza-2′-deoxycytidine, and arsenic trioxide.

19. The method of claim 18, wherein the one or more cytotoxic agents is cisplatin.

20. The method of claim 17, wherein the one or more cytotoxic agents are administered at about the same time as the one or more proteins.

21. A method, comprising killing leukemia cells by contacting the cells with an azurin and a peptide comprising the H.8 region of Laz.

22. The method of claim 21, wherein the leukemia cells are contacted with the azurin and the peptide comprising the H.8 region of Laz at or around the same time.

23. A method, comprising administering to a mammalian patient suffering from leukemia an azurin and a peptide comprising the H.8 region of Laz.

24. The method of claim 23, wherein the azurin and the peptide comprising the H.8 region of Laz are administered to the patient at or around the same time.

25. A method, comprising inducing cellular differentiation in a leukemia cell by contacting the leukemia cell with one or more proteins selected from the group consisting of Laz, azurin, H.8-Azu, Azu-H.8, and the isolated peptide of claim 1.

26. A method, comprising selectively entering cancer cells by contacting the cancer cells with one or more proteins selected from the group consisting of Laz, azurin, H.8-Azu, Azu-H.8, and the isolated peptide of claim 1; wherein the cancer cells are selected from the group consisting of leukemia cells and ovarian cancer cells.

27. A method, comprising inducing cell cycle arrest in a cancer cell by contacting the cancer cell with one or more proteins selected from the group consisting of Laz, azurin, H.8-Azu, Azu-H.8, and the isolated peptide of claim 1.

28. The method of claim 27, wherein the cancer cell is selected from a group consisting of a leukemia cell, a fibrosarcoma cell, a breast cancer, and an ovarian cancer cell.

29. The method of claim 27, wherein the protein increases Wee1 protein levels in the cell.

30. The method of claim 27, wherein the protein causes the depletion of phosphorylated AKT-Ser-473.

31. The method of claim 27, wherein the protein both increases Wee1 protein levels in the cell and causes the depletion of phosphorylated AKT-Ser-473.

32. A method, comprising inducing apoptosis in a cancer cell through caspase 3 activation by contacting the cancer cell with the peptide of claim 1.

33. The method of claim 32, wherein the cancer cell is an ovarian cancer cell.

34. A method, comprising modulating expression of NF-kB signaling pathway genes in a cancer cell by contacting the cancer cell with the peptide of claim 1.

35. The method of embodiment 34, wherein the cancer cell is an ovarian cancer cells.

36. An expression vector encoding the isolated peptide of claim 1.

37. The expression vector of claim 36, which encodes the isolated peptide of claim 4.

38. A pharmaceutical composition comprising the isolated peptide of claim 1.

39. The pharmaceutical composition of claim 38, further comprising a pharmaceutically acceptable carrier.

40. The pharmaceutical composition of claim 38, further comprising a protein selected from the group consisting of Azurin, Laz, H.8-Azu, and Azu-H.8.

41. The pharmaceutical composition of claim 38, further comprising one or more cytotoxic agents that are capable of killing cancer cells.

42. The pharmaceutical composition of claim 38, wherein the pharmaceutically acceptable carrier is appropriate for intravenous injection.

43. A method, comprising administering to a patient suffering from leukemia the pharmaceutical composition of claim 38 in a therapeutically effective amount.

44. The method of claim 43, wherein the pharmaceutical composition is administered to the patient in a manner selected from the group consisting of intravenously, topically, subcutaneously, intramuscularly, orally, and into a tumor.

45. A kit comprising the pharmaceutical composition of claim 38.

46. A nucleic acid molecule, which encodes the isolated peptide of claim 1.

47. A nucleic acid molecule, which encodes the isolated peptide of claim 4.
